(19) United States
(12) Patent Application Publication
(10) Pub. No.: US 2009/0112119 A1
(43) Pub. Date: Apr. 30, 2009

(54) ROTATING BIOPSY DEVICE AND BIOPSY ROBOT

(76) Inventor: Stanley I. Kim, Upland, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/263,107

(22) Filed: Oct. 31, 2008

Related U.S. Application Data
(60) Provisional application No. 61/001,215, filed on Oct. 31, 2007.

(51) Int. Cl. A61B 10/02

(52) U.S. Cl. 600/564

(57) ABSTRACT

Embodiments of a needle biopsy device having a rotating needle mechanism to automatically cut sample tissue that can be operated remotely. A portable small biopsy robot can improve biopsy accuracy, reduce the pain and complications, and shorten the duration of the total biopsy time by using an automatic needle rotating mechanism and a needle localization system that allows a medical practitioner to perform the biopsy procedure from a remote distance. The needle biopsy device can include a cannula and a rotational biopsy needle with a blade the rotational biopsy needle axially and rotatably moveable within the cannula lumen, the blade configured to remove, cut, and/or separate a tissue sample from the target tissue site through rotation of the blade, and to hold the tissue sample in the rotational biopsy needle during proximal retraction from the patient.

### CROSS-REFERENCE TO RELATED APPLICATIONS

This application claims the benefit of priority from U.S. Provisional No. 61/001,215 filed Oct. 31, 2007, which is incorporated in its entirety by reference, herein.

### BACKGROUND OF THE INVENTION

1. Field of the Invention

Various embodiments of the present inventions relate to devices and methods for tissue sampling or excision by means of a biopsy needle that cuts the sample tissue with a rotating movement.

2. Description of the Related Art

When an abnormal lesion such as malignant tumor is found inside an organ such as lung, liver, bone or breast in radiology images such as CT (Computer Tomography) scan, ultrasound scan or mammography, the abnormal lesion needs be sampled to determine its exact nature. Biopsy technique can be varied depending upon the location and the size of the abnormal lesion. In general it can involve a radiology doctor who introduces a needle into the target lesion by puncturing the outer skin while watching the imaging equipment such as CT scan. When the CT scanner is used the doctor has to come in and out of the biopsy room during the biopsy procedure to avoid excessive radiation exposure. Then the doctor generally uses either a very thin needle to aspirate the cells (Aspiration Needle Biopsy) or a thicker needle to cut the core tissue (Core Needle Biopsy) from the target lesion. Some examples of biopsy systems are disclosed in U.S. Pat. No. 4,699,154 Oct. 13, 1987 by Lindgren; U.S. Pat. No. 4,461,305, Jul. 24, 1984 by Cibley; U.S. Pat. No. 6,387,056 B1 May 13, 2002 by Kieturakis; U.S. Pat. No. 6,689,145 B2 Feb. 10, 2004 by Lee, et al.; U.S. Pat. No. 5,944,673 Aug. 31, 1999 by Gregoire et al.; U.S. Pat. No. 6,050,955 Apr. 18, 2000 by Bryan et al.; U.S. Pat. No. 7,189,206 B2 Mar. 13, 2007 by Quick et al; CT-Directed Robotic Biopsy Tested: Motivation and Concept by Cleary et al. Proceedings of SPIE Vol. 4319 (2001); page 231-236; Robopsy-Disposable Robotic Lung Biopsy Assistant by Team Robopsy Hanumara et al. Oct. 9, 2007, which was available at http://www.createthefuturecontest.com/pages/view/entriesdetail.html?entryID=645 in 2007. The Robopsy system described by Hanumara et al. is a biopsy device that is attached onto the patient's body by a strap. The Robopsy systems' mechanism for needle movement uses several different motors that can make the device bulky. It has a mechanical system powered by an electric wire and connected to a laptop computer. Although needle localization can be done by the computer and mechanical engineering system, it is still partially blind biopsy as the radiologist can not see the CT monitor when he or she has to obtain the sample tissue in the last moment of the biopsy procedure. The doctor needs to go back to the biopsy room again, and pull out the stylet and manipulate the needle to cut or such the tissue sample in person without being able to watch the CT monitor. If the biopsy needles have the spring propulsion system, the doctor has to release the trigger, which can cause pain, discomfort and inaccurate sampling by pushing or shaking the biopsy needles and patient's body.

In some aspiration needle biopsy procedures, the aspiration biopsy needle with a stylet will be inserted by puncturing skin to the target lesion while the doctor is watching the imaging monitor screen. Once the needle with the stylet reaches the target area, the stylet will be pulled out to make a space inside the biopsy needle to accommodate the sample tissue or cells that are to be sucked in. The doctor moves, rotates, pulls, or pushes the aspiration needle handle or body slightly to cut, separate or aspirate the sample from the target lesion. Frequently, the doctor has to pull out and reinsert the needle repeatedly, resulting in pain to the patient. Some aspiration biopsy needles can be as thin as 22 gauge for thyroid or lung biopsy while the core biopsy needle as thick as 14 gauge for breast biopsy.

In some core biopsy procedures, the core needle is much thicker as it is intended to obtain a larger chunk of tissue for sampling. It can involve two needles, an outer needle called a cannula, and an inner biopsy needle that has a notch near the distal end. The cannula has sharpened distal opening that severs the tissue prolapsed onto the inner needle notch. Most commonly, a spring propelled mechanism is used to push the cannula over the inner needle for cutting the tissue. This is well illustrated in the U.S. Pat. No. 4,699,154 by Lindgren. There are several problems in common core biopsy techniques.

First, when the cannula and inner biopsy needles are pushed forward to cut the tissue and are separated from the target lesion in cases in which the spring propelled biopsy device is used, often it causes a sudden jerky motion and noise that frightens the patient. It can also cause pain and discomfort.

Second, the forced forward movement of the needle by the spring mechanism can push the needle too far forward to miss the target lesion. This can result in inaccurate sampling that requires repeated biopsy procedures that increase the pain and complications associated with repeated biopsy procedures. The rate of inaccurate or unsuccessful biopsies can be as high as 25% of total biopsy procedures.

Third, when the doctor uses the CT scan as the imaging equipment, the biopsy is a blind biopsy rather than precise image guided procedure, thus often resulting in inaccurate sampling. This is because in order to avoid radiation exposure the doctor has to come in and out of the biopsy room where the patient receives CT scanning during the needle localization procedure. Once the needle localization is complete, the CT scan is turned off, and the doctor then goes back to the biopsy room to trigger the spring loaded biopsy needle device manually. At this very last moment of biopsy procedure, the doctor can not see the last CT scan image to reassure the exactly precise location of the biopsy needle. The needle can be moved again by patient's breathing, coughing or other motion.

The biopsy procedure can be more comfortable and yield more accurate sampling if the manual spring propelled pushing mechanism can be avoided. There are several biopsy procedures that do not use the spring propelled pushing mechanism.

In U.S. Pat. No. 4,461,305 by Cibley, an electric powered rotating mechanism is applied to cut the tissue specimen. However, this technique is not for biopsy of deep seated target tissue inside the organ, but for the sampling of the tissue in the surface, such as uterine cervix. It is more closely related to punch biopsy techniques.

In U.S. Pat. No. 6,387,056 B1 by Kieturakis, rotation of a flexible blade by mechanized power is used. The Kieturakis biopsy system is very complicated, and recovering the severed tissue specimen is often difficult. The blades cutting the tissue are flexible ones that need manipulation. The severed tissues have to be fragmented to be sucked out through the holes by a vacuum mechanism. If the tissue sample is cancerous tissue, cutting the tumor in multiple small pieces can potentially spread the cancer cells within the patient's body. The device ports, lumens and holes may also be blocked if the tissue size is too big to be sucked out.

The U.S. Pat. No. 6,689,145 B2 by Lee, et al. is similar to the U.S. Pat. No. 6,387,056 B1 by Kieturakis with respect to the use of rotating flexible blades that are used to sever the tissue. But it still is complicated to operate and recovering the tissue specimen can also be problematic. It is more suitable for excision of breast tumors rather than for common tissue biopsy using a small sized needle.

In U.S. Pat. No. 5,944,673 by Gregorie et al., the rotating cutting cannula is pushed forward to sever the specimen prolapsed inside the biopsy needle through its apertures. As the cutter is forced to move forward, the whole biopsy needle can be moved or displaced forward also, thus missing the target tissue sampling. In addition, the size of the tissue sample may not be large compared to the size of the needles because of multiple layers of the needles and the suction apparatus. In addition, vacuuming or fluid injection is necessary to obtain the severed tissue sample unless the whole biopsy needle is completely withdrawn. In Gregorie's biopsy system, the rotation of the cutter is done by rotating the knob in the housing manually, not automatically.

In U.S. Pat. No. 6,050,955 by Bryan et al., the biopsy system is similar U.S. Pat. No. 5,944,673 by Gregorie et al. as it involves the rotating forward movement of the cutting cannula, which can push the whole biopsy needle forward, thereby missing the exact target tissue. And the rotation of the cutter is done manually, not automatically. Furthermore, recovering the severed tissue is complicated and difficult as a suction system or fluid and gas injection is required. In U.S. Pat. No. 7,189,206 B2 by Quick et al., the size of tissue specimen obtained may be small relative to the total size of the biopsy needles due to three layers of tubing. A suction apparatus is used to obtain the severed tissue sample.

As described above, standard biopsy procedures that do not use the spring propelled mechanism tend to need to use a suction and vacuum system installed in the biopsy needle to obtain the severed tissue specimen. Certain systems rotate needles with manual manipulation of the knob.

### SUMMARY OF THE INVENTION

Accordingly, there is a need for biopsy procedures that are more comfortable for the patient, easier to use by the medical practitioner, and yield more accurate sampling and excision of tissue for lab work or analysis. There is provided in accordance with one embodiment of the present invention a rotating biopsy device for taking a tissue sample from a target tissue site in a patient body including a cannula and a rotational biopsy needle. The cannula has a lumen and is configured to define an access path to the target tissue site. The rotational biopsy needle has at least one blade. The rotational biopsy needle is axially and rotatably moveable within the cannula lumen. In one embodiment the blade is configured to remove a tissue sample from the target tissue site and to hold the tissue sample in the rotational biopsy needle during proximal retraction from the patient through rotation of the rotational biopsy needle. In one embodiment the at least one blade is configured to remove a tissue sample from the target tissue site through rotation of the at least one blade. In one embodiment the at least one blade configured to hold the tissue sample in the rotational biopsy needle during proximal retraction from the patient.

In one embodiment the rotational biopsy needle also includes a sharp distal head. In one embodiment the at least one blade also includes a first surface and a second surface, with at least one of the first surface and second surface configured to retain a tissue sample. In one embodiment the at least one blade also includes first edge. In one embodiment the rotational biopsy needle further comprises a locking mechanism to releasably lock the rotational biopsy needle position with respect to the cannula. In one embodiment the rotating biopsy device can also include a guide needle. In one embodiment the rotating biopsy device can also include a needle rotator. In one embodiment the needle rotator includes a motor. In one embodiment the needle rotator includes a remote control. In one embodiment the rotational biopsy needle includes a biopsy robot with an adhesive configured to adhere the biopsy robot to the patient's body. In one embodiment the biopsy robot includes a strap configured to attach the biopsy robot to the patient's body.

There is provided in accordance with one embodiment of the present invention a method of collecting a tissue sample from a target tissue site in a body of a patient including inserting a rotational biopsy needle, distally advancing the rotational biopsy needle to a target tissue site in the patient, rotating the rotational biopsy needle, proximally retracting the rotational biopsy needle out of the body. In one embodiment the rotational biopsy needle includes at least one blade. In one embodiment the rotational biopsy needle can be axially and rotatably moveable within a lumen of a cannula. In one embodiment the at least one blade is configured to remove a tissue sample from the target tissue site and to hold the tissue sample in the rotational biopsy needle during proximal retraction from the patient through rotation of the rotational biopsy needle. In one embodiment the at least one blade configured to cut a tissue sample from the target tissue site through rotation of the at least one blade. In one embodiment the at least one blade configured to hold the tissue sample in the rotational biopsy needle during proximal retraction from the patient. In one embodiment the rotating the rotational biopsy needle in a first direction is to remove a tissue sample from the target tissue site. In one embodiment the rotating the rotational biopsy needle step is in a first direction to remove a tissue sample from the target tissue site and to hold the tissue sample in the rotational biopsy needle. In one embodiment the holding a removed tissue sample from the target tissue site is on the at least one blade. In one embodiment the proximally retracting step includes proximally retracting the rotational biopsy needle out of the body of the patient.

In one embodiment the method of collecting a tissue sample also includes inserting the cannula in a patient's body to provide an access path for the rotational biopsy needle. In one embodiment the method also includes locking the cannula to the rotational biopsy needle prior to insertion into the patient's body. In one embodiment the method also includes adjusting the lateral direction of the rotational biopsy needle in a direction orthogonal to the longitudinal axis of the rotational biopsy needle. In one embodiment the method also includes rotating the rotational biopsy needle in a second direction to remove the tissue sample from the rotational biopsy needle. In one embodiment the method also includes attaching a biopsy robot to the patient's body.

In one embodiment the method of collecting a tissue sample of also includes reinserting the rotational biopsy needle to remove an additional tissue sample from the target tissue site. In one embodiment the method of collecting a tissue sample also includes completely excising the target tissue site. In one embodiment the method of collecting a tissue sample also includes rotating the rotational biopsy needle in a second direction opposite the first direction to facilitate the distal advancement of the rotational biopsy needle to the target tissue site. In one embodiment the method of collecting a tissue sample also includes rotating the rotational biopsy needle in a second direction opposite the first direction to remove the tissue sample from the rotational biopsy needle. In one embodiment the method of collecting a tissue sample also includes infusing a material to the target tissue site with an infusion device. In one embodiment the method of collecting a tissue sample also includes treating the target tissue site with a target area treatment device.

There is provided in accordance with one embodiment of the present invention a biopsy robot including a cannula, a rotational biopsy needle, a motor and a controller. In one embodiment the cannula has a lumen and the cannula is configured to access the target tissue site. In one embodiment the rotational biopsy needle has at least one blade. In one embodiment the rotational biopsy needle is axially and rotatably moveable within the cannula lumen. In one embodiment the at least one blade is configured to remove a tissue sample from the target tissue site and to hold the tissue sample in the rotational biopsy needle during proximal retraction from the patient through rotation of the rotational biopsy needle. In one embodiment the at least one blade is configured to separate a tissue sample from the target tissue site through rotation of the at least one blade. In one embodiment the at least one blade is configured to hold the tissue sample in the rotational biopsy needle during proximal retraction from the patient.

In one embodiment the biopsy robot also includes a case bottom with an adhesive configured to adhere the biopsy robot to the patient's body. In one embodiment the controller is controlled from a remote location.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention.

FIG. 1 is a schematic side perspective view of a rotating biopsy device with a needle and cannula according to one embodiment of the present invention.

FIG. 2 is a schematic side perspective view of the rotating biopsy device according to FIG. 1.

FIG. 3 is a schematic side perspective view of the needle according to FIG. 1.

FIG. 4 is a schematic side perspective view of the cannula according to FIG. 1.

FIG. 5 is a schematic side perspective view of the needle distal end according to FIG. 1.

FIG. 6 is a schematic sectional front view of the needle distal end according to FIG. 1.

FIG. 7 is a schematic sectional side view of the needle distal end according to FIG. 1.

FIG. 8 is a schematic side view of a needle according to one embodiment of the present invention.

FIG. 9 is a schematic sectional front view of the needle distal end according to FIG. 8.

FIG. 10 is a schematic sectional side view of the needle distal end according to FIG. 8.

FIG. 11 is a schematic side perspective view of a proximal end of a needle according to one embodiment of the present invention.

FIG. 12 is a schematic side perspective view of a needle rotator according to one embodiment of the present invention.

FIG. 13 is a schematic side view of a rotating biopsy device with a needle, cannula, and needle rotator according to one embodiment of the present invention.

FIG. 14 is a schematic side view of a locking system according to one embodiment of the present invention.

FIG. 15 is a schematic side view of a locking system according to one embodiment of the present invention.

FIG. 16 is a schematic side view of a locking system according to one embodiment of the present invention.

FIG. 17 is a schematic side perspective view of a rotating biopsy device with a needle and cannula according to one embodiment of the present invention.

FIG. 18 is a schematic side perspective view of the rotating biopsy device according to FIG. 17.

FIG. 19 is a schematic side perspective view of the rotating biopsy device according to FIG. 17.

FIG. 20 is a schematic side perspective view of the rotating biopsy device according to FIG. 17.

FIG. 21 is a schematic side perspective view of the rotating biopsy device according to FIG. 17.

FIG. 22 is a schematic side perspective view of the rotating biopsy device according to FIG. 17.

FIG. 23 is a schematic side perspective view of the rotating biopsy device according to FIG. 17.

FIG. 24 is a schematic side perspective view of the rotating biopsy device according to FIG. 17.

FIG. 25 is a schematic side perspective view of the rotating biopsy device according to FIG. 17.

FIG. 26 is a schematic side perspective view of a stylet according to one embodiment of the present invention.

FIG. 27 is a schematic side perspective view of a stylet with a cannula according to one embodiment of the present invention.

FIG. 28 is a schematic side perspective view of a distal end of a needle and a cannula according to one embodiment of the present invention.

FIG. 29 is a schematic side perspective view of the distal end of the needle and a cannula according to FIG. 28.

FIG. 30 is a schematic side perspective view of the distal end of the needle and a cannula according to FIG. 28.

FIG. 31 is a schematic side perspective view of the distal end of the needle and a cannula according to FIG. 28.

FIG. 32 is a schematic side perspective view of the stylet and cannula according to FIGS. 26-27.

FIG. 33 is a schematic side perspective view of the stylet and cannula according to FIGS. 26-27.

FIG. 34 is a schematic side perspective view of the needle and cannula according to FIGS. 28-31.

FIG. 35 is a schematic side perspective view of the needle and cannula according to FIGS. 28-31.

FIG. 36 is a schematic side perspective view of the needle and cannula according to FIGS. 28-31.

FIG. 37 is a schematic front sectional view of a blade with double crescent blades according to one embodiment of the present invention.

FIG. 38 is a schematic front sectional view of a blade with quadruple crescent blades according to one embodiment of the present invention.

FIG. 39 is a schematic front sectional view of a blade with triple crescent blades according to one embodiment of the present invention.

FIG. 40 is a schematic front sectional view of a blade with cross shaped blades according to one embodiment of the present invention.

FIG. 41 is a schematic front sectional view of a blade with double concave blades according to one embodiment of the present invention.

FIG. 42 is a schematic front sectional view of a blade with scooper shaped blades according to one embodiment of the present invention.

FIG. 43 is a schematic perspective side view of a blade according to one embodiment of the present invention.

FIG. 44 is a schematic perspective side view of a blade according to one embodiment of the present invention.

FIG. 45 is a schematic perspective side view of a blade according to one embodiment of the present invention.

FIG. 46 is a schematic perspective side view of a blade according to one embodiment of the present invention.

FIG. 47 is a schematic perspective side view of a blade according to one embodiment of the present invention.

FIG. 48 is a schematic perspective side view of a blade according to one embodiment of the present invention.

FIG. 49 is a schematic perspective side view of a blade according to one embodiment of the present invention.

FIG. 50 is a schematic side sectional view of a biopsy robot according to one embodiment of the present invention.

FIG. 51 is a schematic side sectional view of the biopsy robot according to FIG. 50.

FIG. 52 is a schematic side sectional view of the biopsy robot according to FIG. 50.

FIG. 53 is a schematic side sectional view of a biopsy robot according to one embodiment of the present invention.

FIG. 54 is a schematic side sectional view of the biopsy robot according to FIG. 53.

FIG. 55 is a schematic side sectional view of the biopsy robot according to FIG. 53.

FIG. 56 is a schematic side view of a CT image monitor and a video camera monitor according to one embodiment of the present invention.

FIG. 57 is a schematic perspective view of a biopsy robot on a patient according to one embodiment of the present invention.

FIG. 58 is a schematic perspective view of a biopsy robot according to one embodiment of the present invention.

FIG. 59 is a schematic block diagram of a biopsy robot system according to one embodiment of the present invention.

FIG. 60 is a schematic side perspective view of a rotating biopsy device with a needle and cannula according to one embodiment of the present invention.

FIG. 61 is a schematic side perspective view of the rotating biopsy device according to FIG. 60.

FIG. 62 is a schematic side perspective view of the rotating biopsy device according to FIG. 60.

FIG. 63 is a schematic side perspective view of the rotating biopsy device according to FIG. 60.

FIG. 64 is a schematic side perspective view of the rotating biopsy device according to FIG. 60.

FIG. 65 is a schematic side perspective view of the rotating biopsy device according to FIG. 60

FIG. 66 is a schematic side perspective view of the rotating biopsy device according to FIG. 60.

FIG. 67 is a schematic side perspective view of remaining target tissue after at least one tissue sample removal with the rotating biopsy device according to FIGS. 60-66.

FIG. 68 is a schematic side perspective view of the collapsed remaining target tissue according to FIG. 67.

FIG. 69 is a schematic side perspective view of the rotating biopsy device according to FIGS. 60-68.

FIG. 70 is a schematic side perspective view of the rotating biopsy device according to FIGS. 60-69.

FIG. 71 is a schematic side perspective view of a rotating biopsy device needle according to one embodiment of the present invention.

FIG. 71A is a schematic cross section view of the rotating biopsy device according to FIG. 71.

FIG. 72 is a schematic side perspective view of the rotating biopsy device according to FIG. 71.

FIG. 73 is a schematic bottom perspective view of the rotating biopsy device according to FIG. 71.

FIGS. 74A-C are schematic side perspective views of the rotating biopsy device according to FIG. 71.

FIG. 75 is a schematic side perspective view of a rotating biopsy device with a target area infusion device according to one embodiment of the present invention.

FIG. 76 is a schematic side perspective view of a rotating biopsy device with a target area treatment device according to one embodiment of the present invention.

Throughout the figures, the same reference numerals and characters, unless otherwise stated, are used to denote like features, elements, components or portions of the illustrated embodiments. In certain instances, similar names may be used to describe similar components with different reference numerals which have certain common or similar features. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims. In the following detailed description of some embodiments of the present invention will be given with reference to the drawings. However, the invention is not to be considered as restricted to these embodiments. In addition, the signs in the drawing are not restricted to be used only as marked. For example, the rotation of the needle can be clock-wise, counter clock-wise, or both.

### DETAILED DESCRIPTION

As should be understood in view of the following detailed description, this application is primarily directed to apparatuses, systems and methods for obtaining tissue samples. In one embodiment of the present invention, a rotatable biopsy needle with one or more biopsy blades can be used to effectively cut and obtain tissue samples. In one embodiment a rotating biopsy device with rotating blades cuts a tissue sample and utilizes centrifugal force to hold the tissue in place on the needle for extraction. In one embodiment, the rotating biopsy device uses electric powered automatic continuous rotation for easy recovery of the severed tissue samples. In some embodiments, continuous rotation generates centrifugal force that is very useful to keep the tissue sample in the blades until it is recovered. In one embodiment a total robotic biopsy with a rotating biopsy needle can be used with an automatic and/or remote control system to control localization, rotation of the blades and recovering the severed tissue sample without requiring manual manipulation of the biopsy needle. Furthermore, as the rotating blades can cut the chunk of tissues, it can be used to remove the target tissue completely as a minimal invasive surgical treatment with great accuracy.

In one embodiment a biopsy device comprises an outer cannula, an inner biopsy needle and a needle rotator. The inner biopsy needle has blades in its distal end, and its proximal end is connected to the needle rotator. In one embodiment the needle rotator is powered by electric power. The rotator can be powered by portable DC battery, electric AC power or other source of power. In one embodiment a guide needle localization system can be used with the biopsy device.

In various embodiments biopsy devices, cutting of the tissue sample from the target lesion is accomplished by rotation of the cutting blades located in the distal end of the inner biopsy needle. Once the distal cutting blades are pushed out of the cannula into the tissue of the target lesion, the automatic electric power is turned on either by manual manipulation or by a remote control system to initiate the rotation of the inner biopsy needle. The blades attached in the distal end of the inner biopsy needle rotate, cutting the tissue sample within the target lesion. The speed of rotation can be adjusted depending on the characteristics of the target tissues and the type of the blades. Continuous powered rotation of the blades can keep the severed tissue specimen between the blades by centrifugal force until it is recovered. The continuous rotation that creates the centrifugal force is extremely difficult to achieve by manual rotation of the needle.

Depending upon the location and size of the lesions, different size and shapes of the needle and blades can be used. After the initial cutting of the tissue sample inside the target lesion by the rotating blades, the sample tissue caught between the cutting blades is recovered by withdrawing the inner needle out of the cannula. The inner biopsy needle with the blades can continuously rotate inside the cannula while it is being pulled out to hold the sample in place with minimized contact with the inside of the cannula wall. This helps keep the sample tissue between the blades until it is completely withdrawn and placed in a designated biopsy specimen container. This can eliminate the need of complicated systems of aspiration, suction, vacuum, use of extraneous net, wire, fluid or gas injection to recover the severed tissue samples as described in some of the art.

When the initial sampling is not satisfactory for any reason, the inner biopsy needle having the cutting blades can be reintroduced through the cannula again to obtain the additional tissue specimen. The cannula does not need to be removed each time the needle sample is extracted in repeated or additional biopsy procedures.

Embodiments of the rotating biopsy device have several advantages. First, embodiments of the rotating biopsy device can provide relatively larger biopsy tissue samples than other available biopsy systems. The use of rotating blades instead of a small notch in the inner biopsy needle provides a bigger space to accommodate the bigger tissue sample. In certain embodiments, a blade extends from a small central axis point or core, using most of the diameter of the needle and needle blades as working space for cutting and gathering samples. Notch systems in the art only use a portion of the diameter of the needle or cannula that they may be used with. Embodiments of rotating blades can cut the target tissue in its entirety. In other biopsy systems, only smaller tissue samples relative to the size of the biopsy needles can be obtained due to excessive dead space from the use of a notch in the inner biopsy needle, use of a suction system to recover the tissue sample, or multiple layers of the needles used therein.

Second, embodiments of the rotating biopsy device can be much more comfortable to the patient because cutting the tissue by gently rotating blades does not cause the sudden jerky motion associated with the movement of a needle pushing into tissue by the sudden release of a compressed spring such as is used in the spring propelled biopsy systems. The size of the needle can be made very thin or small because of its simplicity of the biopsy device. Certain embodiments of the rotating biopsy device are not very complicated apparatus. The inner biopsy needle can be thinner than 22 gauge with very small cutting blades. It can replace the current fine needle aspiration biopsy procedure while providing more tissue sample with higher accuracy. The thinner the biopsy needle, the less the chance of pain and complication.

Third, embodiments of the rotating biopsy device can improve the probability of accurate tissue sampling of the target lesion. For example, one embodiment of the rotating biopsy device does not use a spring propulsion mechanism to cut the tissue. The sudden jerky motion associated with the release of the compressed spring to push the biopsy needles to cut the tissue can displace the initial needle localization, thus missing the accurate tissue sampling. In addition, the capability of using remote control system to turn on the electric powered rotator can allow the doctor to watch the CT or other imaging equipment right at the moment of the cutting the tissue, further improving accuracy of the tissue sampling.

Fourth, embodiments of the rotating biopsy device can eliminate the need of complicated biopsy equipment which can be very costly and bulky. Embodiments of the rotating biopsy device are easy to manufacture and simple to operate. Like an automatic screwdriver rotator, the biopsy needles can be removed and inserted very easily by pushing in or pulling out of the needles from the connection site of a rotator. This simple system can allow many small low budget hospitals in the world to perform the necessary biopsy procedures at a lower cost.

Fifth, the automatic rotating mechanism used in embodiments of the biopsy system can make remote controlled robotic biopsy possible. A very small robot system can control the biopsy needles and the blades while the doctor manipulates the portable robot attached onto the patient in a separate control room watching the image monitor screen. The total duration of time spent for the biopsy procedure is shortened because the radiology doctor does not need to walk in and out to the biopsy room during the biopsy procedure. The portable robot inserts the biopsy needle into the target lesion, rotates the biopsy needle severing the tissue, and recovers the tissue sample automatically. This function not only can prevent radiation exposure to the radiologist doctor but also help the doctor localize the biopsy needle and obtain the tissue sample in a great accuracy.

Sixth, the whole biopsy procedure can be done from a safe the distance using a wired or wireless remote system to control and/or power the biopsy robot. The doctor could be in the same treatment area as the patient, or can be remote from another room in a hospital, or in another location miles away or even on the other side of the world. Using a telecommunication network system, the doctor can perform the robotic biopsy procedure in a different place far away from the patient. The portable robot has the functions of needle localization and rotation of the biopsy needle to cut the tissue sample by wireless remote system. By controlling the biopsy robot, the radiologist doctor can perform the true automatic hand free biopsy procedure while watching the CT or other imaging equipments in the remote distance. For example, with some assistance by a technician or nurse, a doctor in a city can perform the biopsy for a patient in a rural hospital using the sample portable biopsy robot, portable digital imaging equipment, camera and remote control system.

Seventh, the risk of an accidental tissue injury associated with sudden movement of the patient during the procedure is less with a robotic biopsy system that can be made in a small portable size that is easily attached on to the patient's body near the biopsy site. Because of its smaller size that can be attached onto the skin near the biopsy site, it drastically reduces the risk of accidental injury due to needle breaking associated with patient's sudden motion. When the patient moves due to breathing or even coughing, the whole biopsy robot system with needles will move as one with the patient's body, which is far different from the robotic biopsy system in the prior art by Cleary, et al in an article ‘CT-Directed Robotic Biopsy Tested: Motivation and Concept’. In the art, some robotic arms may be used to hold the biopsy needle, which can be connected to fixed equipment. When the patent moves, the biopsy needle held by the fixed robotic arm can not move accordingly, thus increasing the risk of needle dislocation and injury. Even with a separate sensor detecting the patient's motion, it can not be as natural as this system attached onto the patient's skin.

Secure coupling or tight attachment between an embodiment of the biopsy robot and the patient can be achieved by using adhesives placed on the bottom of the biopsy device. The strong adhesives keep the biopsy device adhered to the patient's skin tightly and firmly. In one embodiment the biopsy robot is further reinforced by a strap from the biopsy device wrapping portions of the patient's body. However, solely wrapping a biopsy device to a patient's body with only a strap may not be enough to hold the biopsy device on the patient's body. In one embodiment of a robotic biopsy device, the bottom of the biopsy device is treated with strong adhesives that can attach the device tightly to the patient's body. The wrapping the patient body by the strap can further reinforce the tight attachment of the biopsy robot to the patient. Therefore, the patient and the biopsy device move as one body, which eliminate the risk of needle breaking or other needle injury. It also eliminates the need of extra sensor systems to monitor relative body movement.

Eighth, the biopsy robot can be used as a surgery robot to excise the target tissue completely rather than taking a small sample. With the rotating blades that can cut the tissue in its entirety, a complete excision of a lesion can be done. By using the different cutting blades and by examining the excised tissues by a pathologist at the premise, the complete excision of the target lesion can be confirmed. It is particularly useful to remove the target lesions located deep inside of the body with minimal invasiveness and tissue injury. If the target lesion is cancerous, chemotherapeutic drugs can be injected through the cannula before and after the excision to ensure complete elimination and eradication of the cancer cells in the excision area. Anesthetic drugs such as lidocaine, or vaso-constricting agents such as epinephrine can be administered before or after the tissue sampling to numb the inner site or to control bleeding if necessary. The sample tissue severed by the rotating blades can be obtained easily without using complicated systems as described in the art, such as aspiration, suction, vacuum, net, wire, fluid or gas injection. Automatic continuous rotation of the blades creates centrifugal force that can keep the tissue sample in the space between the rotating blades until it is recovered and placed in the designated biopsy tissue container.

In various embodiments, a biopsy system can be used to biopsy the tissue sample from various sites, such as prostate, breast, brain, bone, bone marrow, lung, liver, kidney, or even heart, with slight modification of the size, shape, configuration or length of the needles and the cutting blades. For example, in case of prostate biopsy, multiple thin needles attached to the needle rotator can be inserted and then rotated simultaneously obtaining the tissues while watching the ultrasound images. Currently, a total of roughly 6-8 needles punctures are made sequentially for prostate biopsy. In the spring propelled system used for the current prostate biopsy, the needles often move forward beyond the prostate capsule, thus injuring the urethra and adjacent organs causing severe pain, bleeding and infection. Each puncture can cause pain and those complications. In case of breast biopsy, by using the remote control system, MRI image guided biopsy can be done. The breast biopsy under mammography visualization, the so-called stereotactic breast biopsy, can be done without complicated biopsy equipment or system. In case of bone marrow biopsy, the bone marrow tissue can be obtained without any chance of losing the severed bone marrow core. In case of bone biopsy, the distal tip of the inner biopsy needle can be modified to have helical or spiral threads on the surface of its conical tip in order to penetrate the hard bone surface. Currently radiologist doctors use a hammer to penetrate the hard bone surface to obtain bone sample. With the rotating mechanism, bone biopsy can be accomplished very easily with a threaded configuration.

Various materials can be used in various embodiments of the present invention. Although stainless steel is used commonly for most of the biopsy device, non-iron containing materials such as titanium or hard synthetic materials can be used to provide clear images when magnetic resonance (MR) imaging equipment is used. For reducing the size and the weight of the device, certain parts of this device can be made of different materials. For example, composite or plastic materials can be used. In one embodiment plastic is used for the frame or case of the device.

Embodiments of the parts of this invention can be of any size, shape or configuration. For instance, the biopsy needle for lung biopsy is smaller than that for breast biopsy. The cutting blades can have various shapes and configuration that are not restricted to the ones in the drawings. In some embodiments, one or more types of lubricating materials can be used with the parts involving rotation. The parts in embodiments of this invention can be used with or without other components. For example, the structure used for the biopsy robot including the frame, the top, the bottom with its adhesives treated extension and the strap can be used to support the needle rotator alone when the doctor does the biopsy semi-automatically. Any of the embodiments of the rotating biopsy device 1 disclosed herein can have features or aspects similar or identical to other embodiments, along or in various combinations. For example, any aspects of a blade, edge, rotational feature, and other characteristics of various embodiments may be employed or used with other embodiments herein.

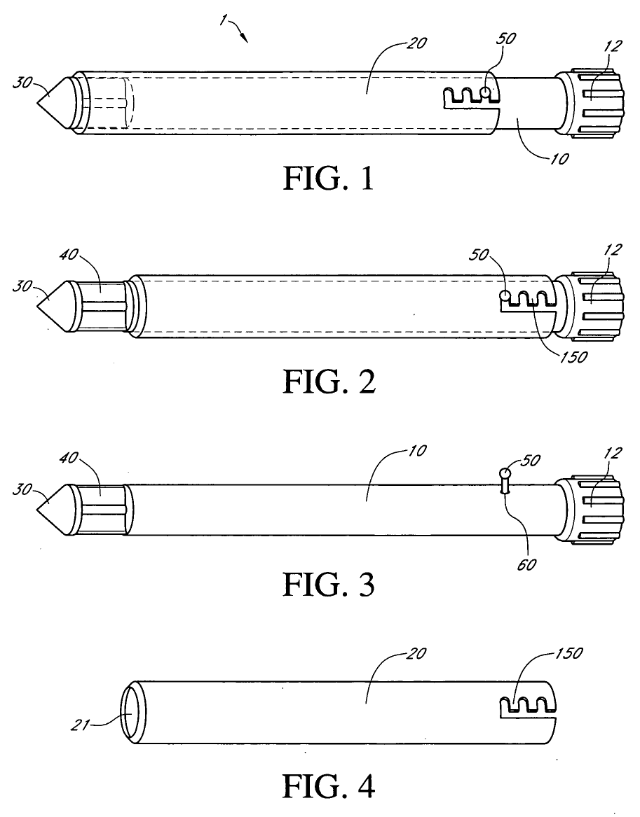

FIGS. 1-4 illustrate one embodiment of a rotating biopsy device 1 with an inner biopsy needle 10 insertable into a cannula 20. One embodiment of the cannula 20 has a lumen 21 extending along a longitudinal axis of the cannula 20. In one embodiment, the cannula 20 is sized and configured to extend from a target tissue on or inside the body of a patient to a position outside the patient, providing a lumen between the target tissue and the outside of the body. One embodiment of the inner biopsy needle 10 has a cone-shaped sharp head 30 at the distal end. The cone-shaped head 30 can be used like a trocar or stylet to help move or pierce tissue at the distal end of the cannula 20 in order to access a sample site. In one embodiment the needle 10 has a blade 40. In various embodiments, the blade 40 is located in a distal region of the needle 10. In one embodiment the blade 40 is configured to cut, sever, remove, or separate a tissue sample from the target tissue site through rotation of the blade 40 or the rotation of the rotational biopsy needle 10. In one embodiment the blade 40 is configured to hold the tissue sample in the rotational biopsy needle during proximal retraction from the patient. In one embodiment the blade 40 is configured to hold a tissue sample in a specific axial position, or constant axial position at a distal region of the rotational biopsy needle. In one embodiment the axial position at which the blade 40 holds the tissue sample is constant with respect to the needle 10, with the needle 10 moveable with respect to a cannula 20. In one embodiment the blade 40 is configured to cup a tissue sample against a first surface of the cutting blade using centrifugal force generated by the rotation of the needle 10 in a first rotational direction. In one embodiment the blade 40 is configured to release a tissue sample from a first surface of the cutting blade using centrifugal force generated by the rotation of the needle 10 in a second rotational direction. In one embodiment the rotational device 1 can be configured to capture and retain a tissue sample on a second surface.

In various embodiments, the blade 40 can be have a length that can be of various sizes depending on the size, shape, material, and condition of the tissue sample of interest. The blade 40 diameter can extend to approximately the inner diameter size of the cannula 20. The core at the rotational axis of the blade 40 can have a diameter with sufficient strength and rigidity to hold the blades and head 30 in place, but small enough to gather a sufficient tissue sample size. In one embodiment the proximal end is configured to connect the needle to a rotator, as illustrated in one embodiment in FIG. 12.

In one embodiment the conical sharp head 30 can have the spiral threads on its surface to penetrate a hard tissue such as bone surface. A screw-shaped head may be better than the smooth conical head for bone penetration.

In one embodiment the inner biopsy needle 10 has a locking mechanism, such as a locking pin 50 that is inserted into the hole 60 located near the proximal end of the inner biopsy needle 10. The pin 50 is locked into the gear or slot 150 of the cannula 20 to keep the inner biopsy needle 10 and the cannula 20 from the moving separately during the insertion or other steps in which the needle 10 and cannula 20 are kept together.

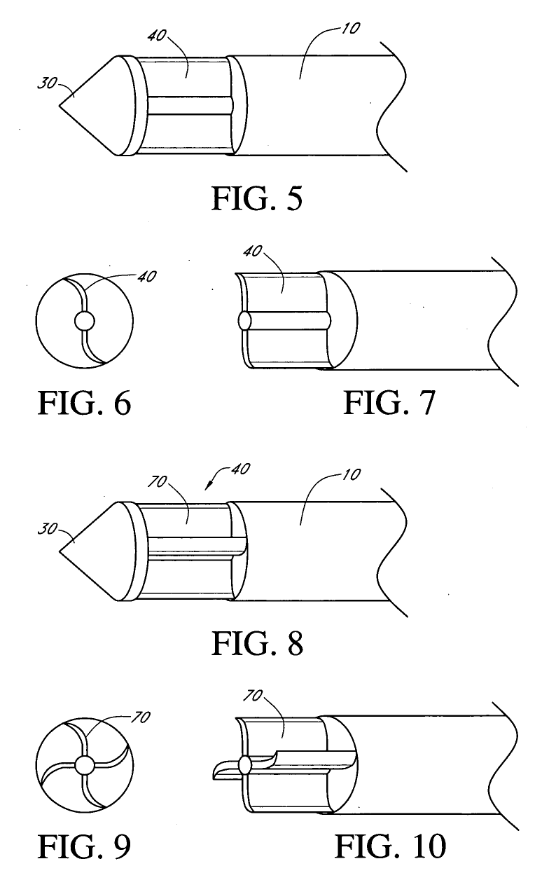

FIGS. 5-7 are perspective detailed views of an embodiment of the distal end of the inner biopsy needle 10 having the conical sharp end 30 that penetrates to the target tissue through the outer skin and the double winged cutting blades 40. FIG. 6 is a cross-sectional anterior view and FIG. 7 is a cross-sectional view of the double winged cutting blades 40. FIGS. 8-10 are the perspective view of another embodiment of the distal end of the inner biopsy needle having cutting blades 40 in a quadruple winged cutting blade 70 embodiment. FIG. 9 is the cross-sectional anterior view, and FIG. 10 is the cross-sectional lateral view of the quadruple winged cutting blades 70.

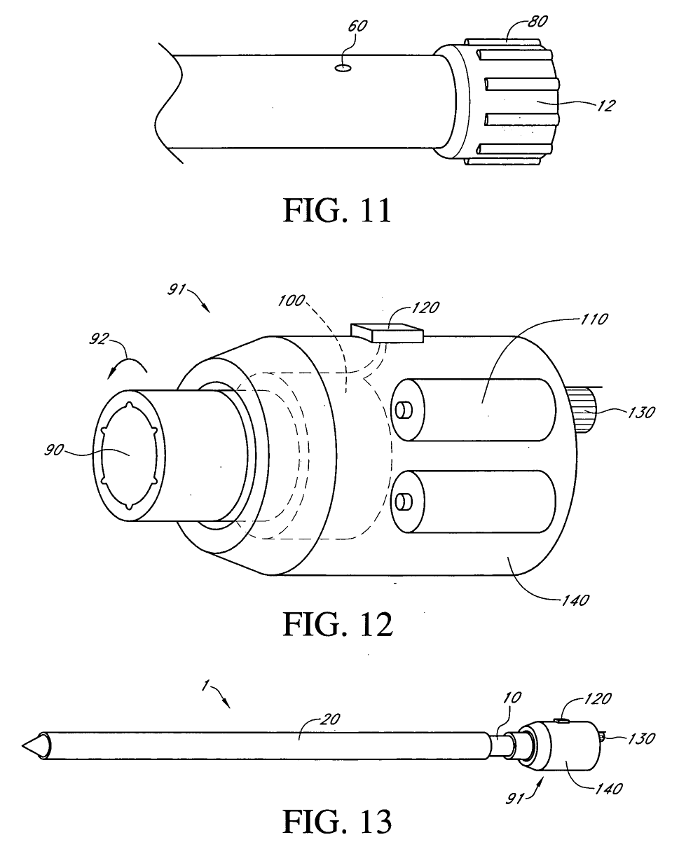

FIG. 11 is a perspective detailed view of an embodiment of the proximal end of the inner biopsy needle 10 having the hole 60 for the locking pin 50, and the connecting part 12 at the end that is to be connected to the needle rotator of FIG. 12. In one embodiment the connecting part 12 has one or more longitudinal protuberances 80 configured to attach (for a tight fit) to the rotator's needle receptor 90. In various embodiments various keyhole configurations or shapes can be used to rotate the needle 10 with a needle rotator.

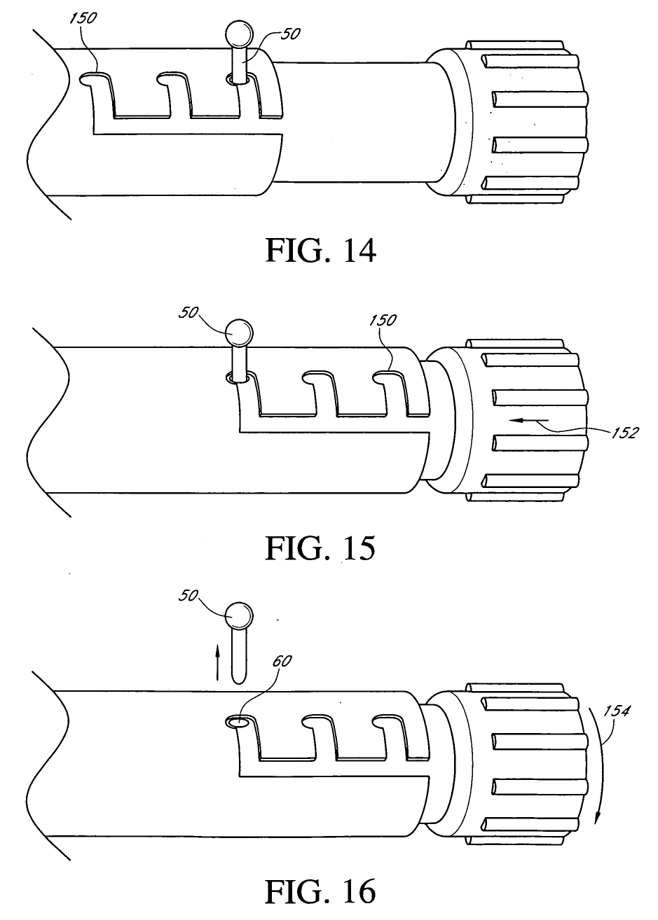

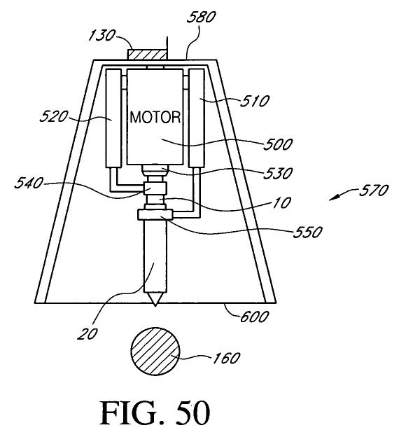

FIG. 12 is a perspective view of an embodiment of a needle rotator 91 having the needle receptor 90 operatively connected to a motor 100. In one embodiment the needle rotator 91 can comprise a biopsy robot with a motor 500, as described with respect to FIGS. 50-59. In one embodiment, the motor 100 is powered by one or more batteries 110. In one embodiment the needle rotator 91 has a push button 12 that turns on the power to rotate the needle 10. Rotation of the needle receptor 90 is shown as a counter-clockwise arrow 92 as viewed from the front of the needle rotator 91, however, the rotation can be clockwise in an embodiment. In one embodiment a wired transmitter controls axial and rotational movement of the needle 10. In one embodiment a wireless transmitter and remote control receiver 130 is attached to operate the rotator by remote control. The rotator can be very small in size and light in weight using a very small sized motor and battery. The small sized rotator can be easily attached to the biopsy needle without distorting the needle. In one embodiment the receptor 90, motor 100 and battery 110 are encased in the rotator case 140. FIG. 13 is a perspective view of one embodiment of a rotating biopsy device 1 comprising a needle 10, cannula 20 and a needle rotator 91.

FIGS. 14-16 are perspective detailed views of one embodiment of a locking system that locks the cannula 20 and inner biopsy needle 10. In one embodiment the locking system locks the cannula 20 and inner biopsy needle 10 at specific axial locations with respect to each other while limiting rotation with respect to each other. In one embodiment, when the inner biopsy needle 10 is the “pre-rotation” mode, it is locked into the cannula by the locking pin 50 inserted to the pin hole 60 on the surface of the inner biopsy needle 10 in the first row of the slot 150 of the cannula 20. When the inner biopsy needle 10 is pushed forward in the “ready to rotate” mode, the locking pin 50 is moved to the third row of the slot 150. The forward movement of the inner biopsy needle can be set in the middle row of the slot 150 if necessary. The pin 50 can be removed from the hole 60 to allow for rotation of the needle 10 with respect to the cannula 20. In one embodiment the locking system has the similar look of the gear box of a car. Axial motion to extend the needle 10 distally is represented by the arrow 14. Retraction in the proximal direction would be in the opposite direction of arrow 14, such as is illustrated by arrow 18. Rotation of the needle 10 is represented in one embodiment by the counterclockwise arrow as viewed from the front end from distal perspective of the needle by the arrow 16. In various embodiments, the blades 40 can be configured to cut in a clockwise or counterclockwise direction. In some embodiments, the blades 40 can be configured to release sample tissue material by rotating in a direction opposite a cutting rotation direction.

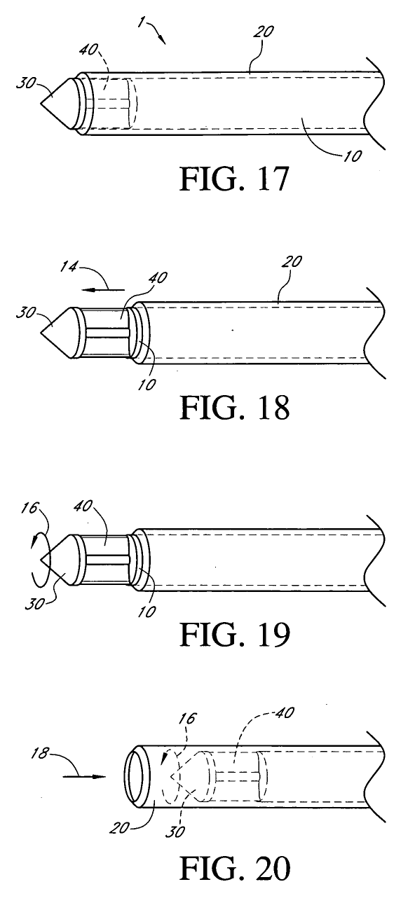

FIGS. 17-20 are perspective views of one embodiment of a rotating biopsy device 1 in operation. FIG. 17 is a perspective view of the rotating biopsy device 1 in the “pre-rotation” mode or configuration. The distal end 30 of the needle 10 can be placed in a proximal end of the cannula 20 and advanced distally such that the distal end 30 extends beyond the distal end of the cannula lumen 21. FIG. 18 is a perspective view of the device 1 in the “ready to rotate” mode in which the distal end of the inner biopsy needle 10 can be pushed forward or distally into the target tissue. The cutting blades 40 can then be exposed to the tissue of the target lesion. FIG. 19 is a perspective view of the device 1 in “rotating” mode or configuration. The needle rotator 91 is turned on to rotate the needle 10, and the cutting blades 40 are rotating continuously along with the inner biopsy needle 10. The tissues of the target lesion are severed and caught in the space between the blades. Centrifugal force generated by the continuously rotating blades helps retain the tissue sample on the rotating blades until it is recovered at a later stage of the biopsy procedure. FIG. 20 illustrates the rotating blades 40 and the inner biopsy needle 10 being pulled proximally through cannula 20, and the needle 10 can be fully extracted proximally from the cannula 20.

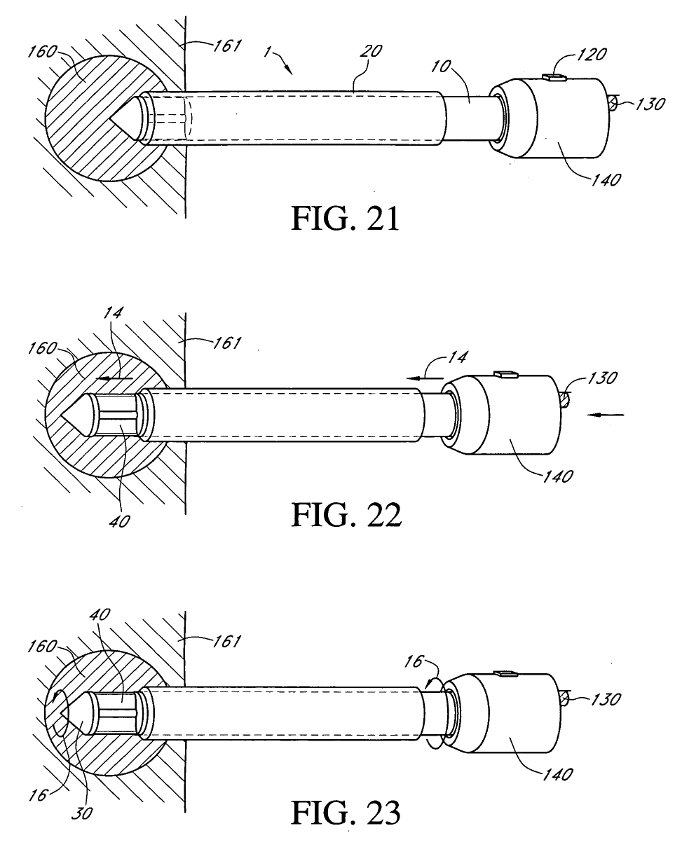

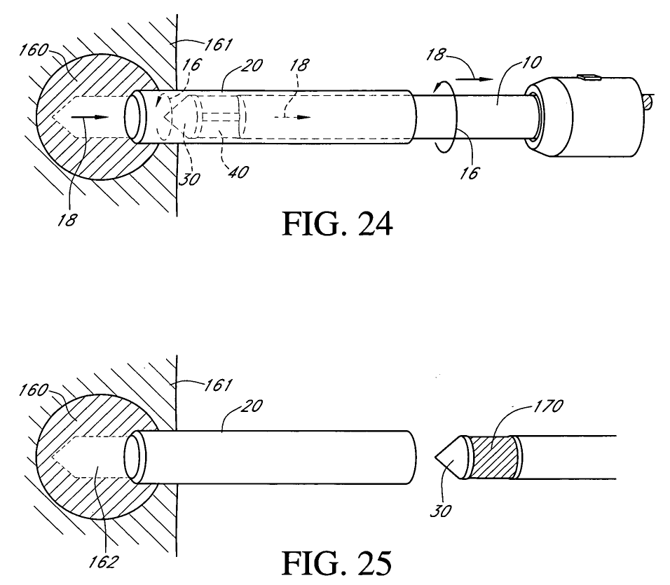

FIGS. 21-25 illustrate one embodiment of a rotating biopsy device 1 in operation. FIG. 21 shows the cannula 20 and the inner biopsy needle 10 are inserted into the target lesion 160. The target lesion 160 may be located at a surface of tissue 161, or may be located or enclosed within a region of tissue 161. In various embodiments, the rotating biopsy device 1 is configured with a length and size sufficient to operate the rotating biopsy device 1 and have it extend to a target lesion 160 of interest. The deeper the target lesion 160 is located within the tissue 161, the longer the rotating biopsy device 1 can be. FIG. 22 shows the cutting blades 40 in the distal end of the inner biopsy needle 10 as they are pushed forward distally into the target lesion 160. FIG. 23 shows the cutting blades 40 are rotating along with the inner biopsy needle 10 inside the target lesion 160, thus cutting the tissue sample 170. The rotation continues as long as the rotator is activated. In one embodiment the rotation continues as long as the rotator button 120 is being pressed. The wireless transmitter and remote control receiver 130 and its remote controller can be used to operate the rotator at a distance. FIG. 24 shows that the rotating cutting blades along with the inner needle are pulled backward inside the cannula 20, leaving a cavity 162 within the target tissue 160. In one embodiment, the entire target tissue 160 can be cut and removed by the rotating biopsy device 1, leaving a cavity 162 in the tissue 161. FIG. 25 shows that the inner biopsy needle 10 is completely pulled out of the cannula 20, and the tissue sample 170 is recovered for a pathology examination.

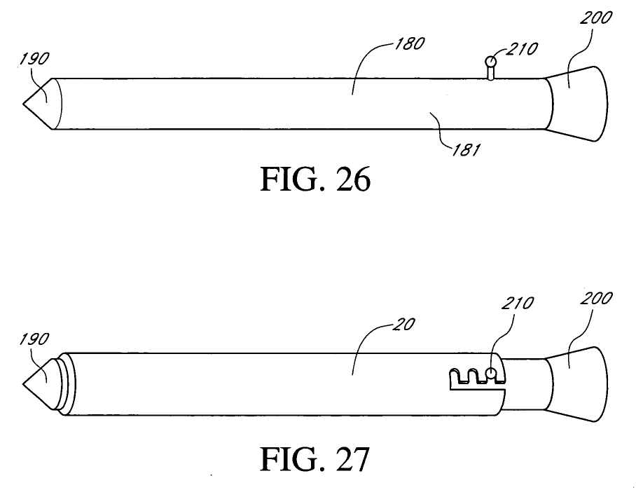

In one embodiment of a rotating biopsy device 1, a stylet 180 can be used to pierce or to help direct the rotating biopsy device 1 to a target tissue 160. FIG. 26 is a perspective view of one embodiment of a stylet 180 that can be used as a guide needle along with the cannula 20 in a different embodiment of the rotating biopsy device 1. In one embodiment the stylet 180 has a distal end 190, a body 181 and a proximal end handle 200. In various embodiments the stylet distal end 190 can be sharp, cone-shaped, atraumatic, blunt, solid, malleable, and/or threaded. In one embodiment a locking pin 210 is located near the proximal end handle 200. The locking pin 210 can be detachable if the guiding stylet 180 needs to be rotated with respect to the cannula 20, such as to penetrate hard tissue. The conical end 190 also can have the spiral threads for easier rotating penetration. FIG. 27 is the perspective view of the guide needle system having the stylet 180 inserted into the cannula 20 in the locking mode.

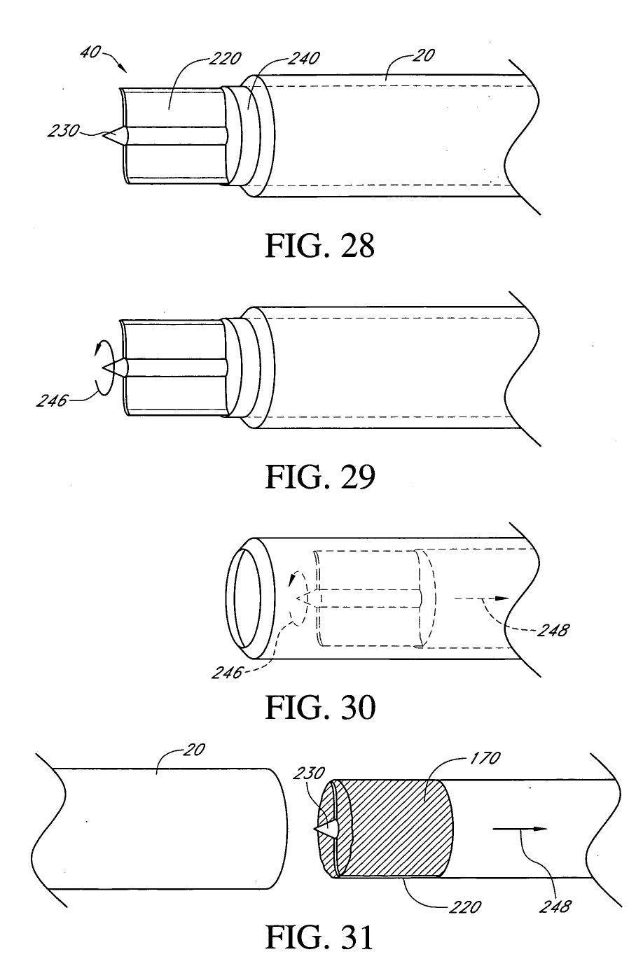

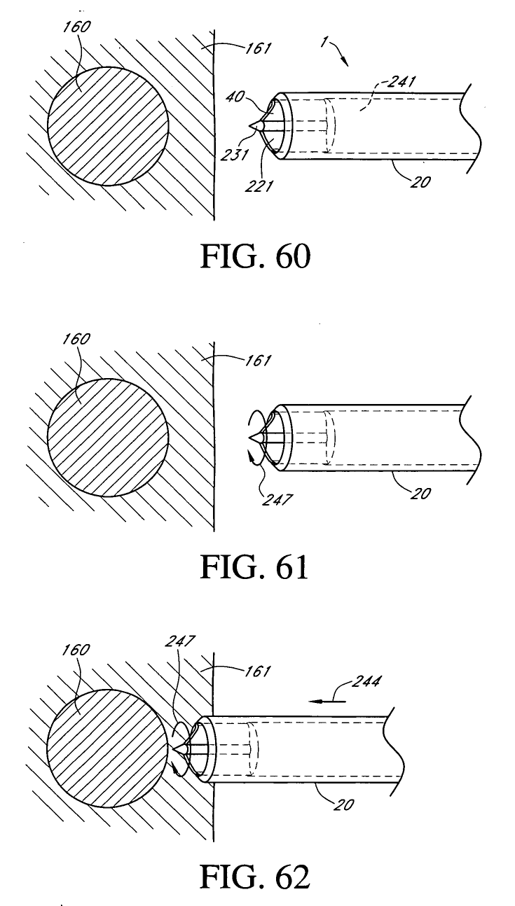

FIGS. 28-31 are perspective views of an embodiment of a rotating biopsy device 1 with a rotating biopsy needle 240 with a stylet 180 or guide needle system as shown in FIGS. 26-27. FIG. 28 is a perspective detailed view of an embodiment of cutting blades 40 in an open end cutting blade 220 embodiment with a sharp conical end 230. In various embodiments, any cutting blades can be same or similar to embodiments of cutting blades 40, with one or more blades and in various combinations of embodiments. In one embodiment a distal portion of the blade 220 can have a roughly square edge. In one embodiment a distal portion of the blade 220 can be slightly sloped, as is illustrated in FIG. 60 blade 221. The shape and configuration of the blades can be various depending on the type of biopsy or tissue excision. In various embodiments of blades disclosed herein, surfaces, edges, and/or other features can be applied to various locations. For example, embodiment of a cutting edge or dull edge can be located on any edge that contacts tissue. Thus, a cutting edge or dull edge or other feature can be on the circumference of a blade, along the distal/leading edge of a blade, internal to a blade, along the proximal/tail edge of a blade, or wherever the blade comes in contact with tissue. FIG. 29 is a perspective view of the blades 220 rotating in a direction denoted by 246. In one embodiment the rotation can be in the opposite direction. FIG. 30 shows the rotating blades of the inner biopsy needle 240 are being pulled backward proximally to recover the severed tissue sample. FIG. 31 shows the blades 220 completely pulled out of the cannula 20, with the cutting blades 220 having the tissue sample 170.

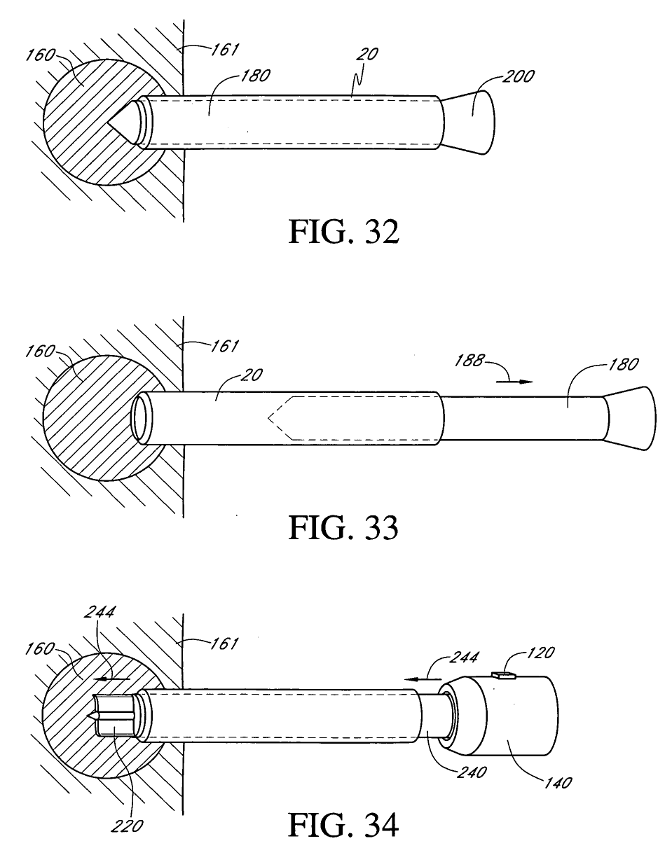

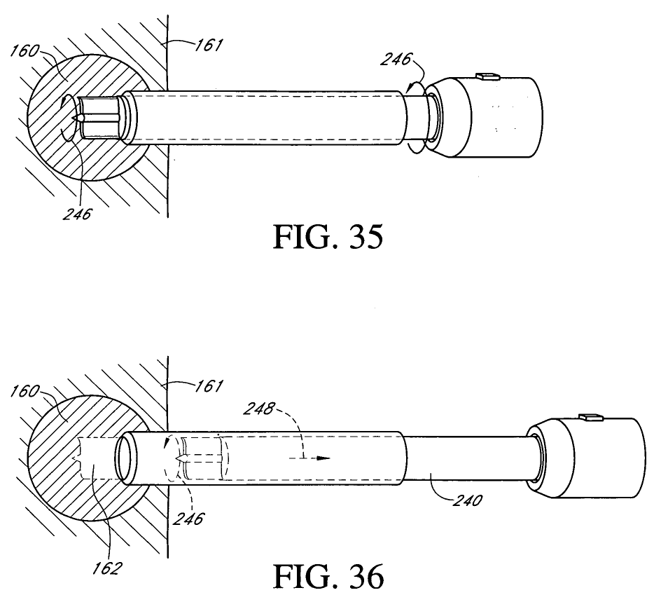

FIGS. 32-36 illustrate steps in one embodiment of the use of a rotating biopsy device 1 with a needle 240 using a guide needle system with a stylet 18. FIG. 32 shows the guide needle system consisting of the cannula 20 and the stylet 180 inserted into the target lesion 160. FIG. 33 shows the stylet 180 being pulled out of the cannula 20 once the localization of the cannula 20 is satisfactory for the biopsy. FIG. 34 shows the inner biopsy needle 240 is inserted into the target lesion 160 by extending distally through cannula 20, which has already been placed in the target lesion 160. FIG. 35 shows the rotating blades 220. In various embodiments the push button 120, wired transmitter, or wireless transmitter and remote control receiver 130 are used to turn on and operate the needle rotator 140. FIG. 36 shows the inner needle 240 is being extracted proximally out of the cannula 20 to recover the tissue sample 170.

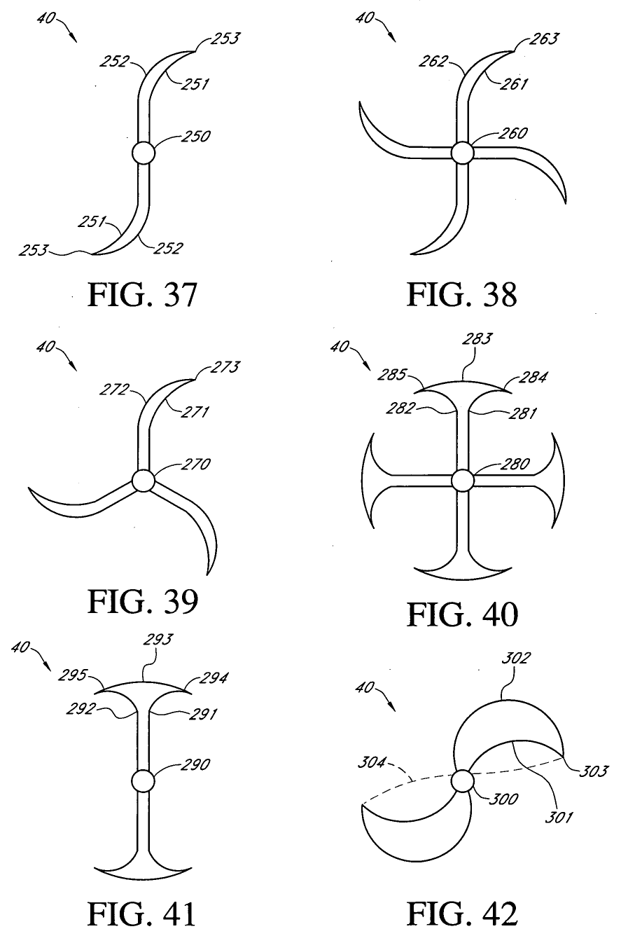

FIGS. 37-42 illustrate some embodiments of cross-sectional views of cutting blades 40. The shape and configuration of the blades can be modified as necessary for the biopsy procedures of various location, depth and size. In various embodiments, the blade 40 is attached to a core at or near the axis of rotation of the needle 10. A larger volume of target tissue sample can be taken with a blade 40 with a relatively smaller core, providing for more surface area and exposing a relatively larger cutting profile in the target tissue 160. In addition, any combination of any shape and configuration can be used. In one embodiment the blade 40 can be configured to cut tissue with rotation in a first direction, either clockwise or counterclockwise. In one embodiment the blade 40 can be configured to cut tissue with rotation in a second direction, opposite the first direction. In one embodiment the blade 40 can be configured to move or displace tissue without cutting it with rotation in a second direction, opposite the first direction. Variations in speed of rotation can also have various effects on the cutting action of various embodiments of the blade 40.

In various embodiments of blades 40, edges may be illustrated with a surface or edge shown in cross-section. However, in some embodiments the edge or surface feature may exist along another edge or surface not shown in cross-section or side view. For example, embodiments of some blades 40 have an exposed distal end that is flat, straight, tapered, sloped or other wise disposed to include a cutting edge along the exposed distal edge. Various embodiments of aspects of blades, edges, surfaces and other features can apply to any edge, on the side, front, back, or any exposed aspect of the blade as well. FIG. 37 shows an embodiment of the double crescent shaped blade 250 with a central core, first blade surface 251, second blade surface 252 and cutting edge 253. FIG. 38 shows an embodiment of the quadruple crescent blade 260 with a central core, first blade surface 261, second blade surface 262 and cutting edge 263. FIG. 39 shows an embodiment of the triple crescent blades 270 with a central core, first blade surface 271, second blade surface 272 and cutting edge 273. FIG. 40 shows an embodiment of the blades of the Maltese Cross 280 with a central core, first blade surface 281, second blade surface 282, outer surface 283, first edge 284 and second edge 285. FIG. 41 shows an embodiment of a double concave blade 290 with a central core, first blade surface 291, second blade surface 292, outer surface 293, first edge 294 and second edge 295. FIG. 42 shows an embodiment of an ice cream scooper style blade 300 with a central core, first blade surface 301, second blade surface 302 and cutting edge 303. Line 304 indicates a slope or helical surface on the first blade surface 301. In one embodiment scooper blade 300 is similar to an ice cream scooper shape. In one embodiment the blades 40 are configured to cut and retain tissue samples when rotating in a first direction. In one embodiment the blades 40 are configured to release tissue samples when rotated in a second direction.

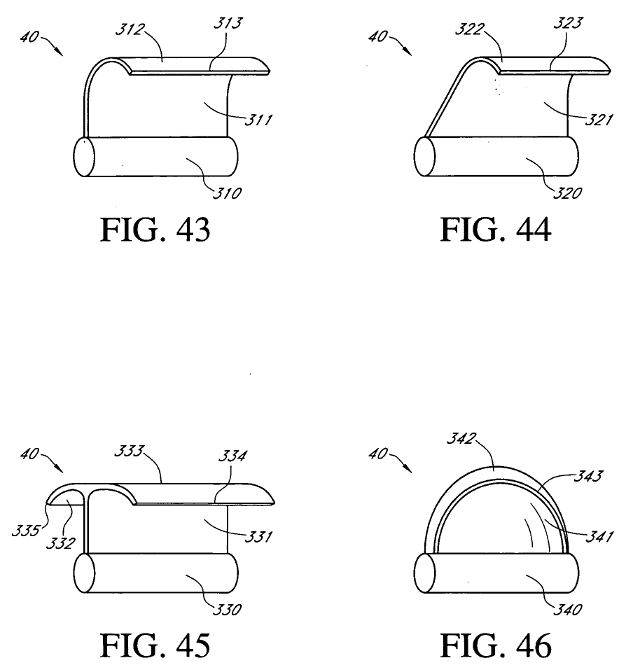

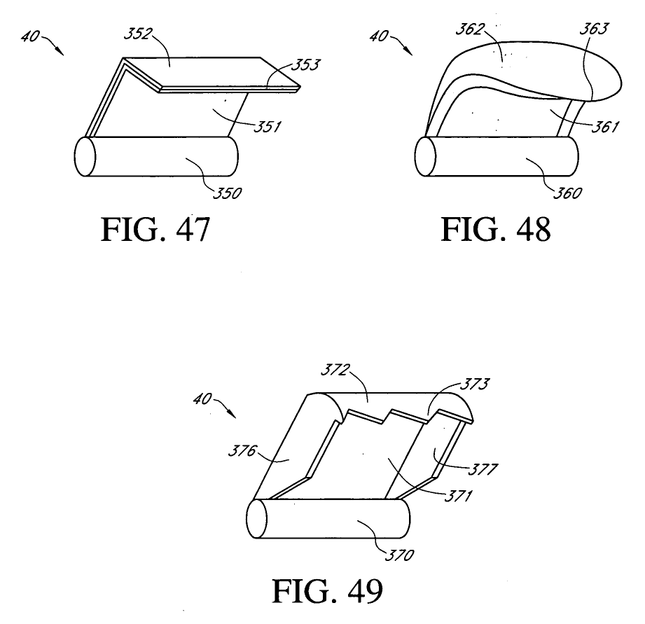

FIGS. 43-49 illustrate single blades from some embodiments of cutting blades 40. Any of these blades 40 can have a plurality of blades, even if only one is illustrated. The shape and configuration of the blades can be modified as necessary for the biopsy procedures of various location, depth and size. In addition, any combination of any shape and configuration can be used. In one embodiment the blades 40 are configured to cut and retain tissue samples when rotating in a first direction. In one embodiment the blades 40 are configured to release tissue samples when rotated in a second direction. FIG. 43 shows an embodiment of the curved rectangular shaped blade 310 with a central core, first blade surface 311, second blade surface 312 and cutting edge 313. FIG. 44 shows an embodiment of the blade having a slanted side 320 with a central core, first blade surface 321, second blade surface 322 and cutting edge 323. FIG. 45 shows an embodiment of the blade having double wings 330 with a central core, first blade surface 331, second blade surface 332, outer surface 333, first edge 334 and second edge 335. FIG. 46 shows an embodiment of the blade of the shape of a half dome 340, such as an ice cream scooper. In one embodiment the half dome blade 340 comprises a curved blade orthogonal to the rotation axis 340 with a central core, first blade surface 341, second blade surface 342 and cutting edge 343. FIG. 47 illustrates an embodiment of a blade with an angular surface 350 with a central core, first blade surface 351, second blade surface 352 and cutting edge 353. In one embodiment the angular surfaces are at roughly a right angle. FIG. 47 shows an embodiment of a curved blade 360 having a core, first blade surface 361, second blade surface 362, rounded cutting edge 363 and one or more side walls. Although not illustrated in FIGS. 43-47, any embodiment of the blades 40 can have one or more side walls. In some embodiments the side walls are provided by the shaft of the needle 10 or 240, or by the distal tip 30. FIG. 48 shows an embodiment of a rectangular blade 370 having a first blade surface 371, second blade surface 372, serrated edge 373 and one or more side walls 376, 377.

The materials for the blades and the parts of the biopsy device 1 also can be of metals, hard plastics, or others that are hard enough to penetrate the skin, mucous membrane or inner organs. For certain occasions such as magnetic resonance image guided biopsy, the material of the biopsy device is free from ferrous, ferric or other iron molecules for a better resolution and clear images.

FIGS. 50-52 illustrate an embodiment of a biopsy robot system. In various embodiments, the biopsy robot 570 can attached to a patient to perform biopsies in a remote, automated, convenient manner. In various embodiments the biopsy robot can use portable battery power, AC electric power or some other power source. In one embodiment the biopsy robot has one or more motor units 500 that are configured to control the axial and/or rotational movement of a needle 10. In one embodiment the biopsy robot has one or more motor units 500 that are configured to control the axial and/or rotational movement of a cannula 20. In one embodiment the biopsy robot is configured to control the axial, rotational, and/or lateral positioning of the needle 10 and/or cannula 20. In one embodiment the biopsy robot is configured to control and adjust lateral directional positioning axis of the needle 10 and/or cannula 20 along a X-axis orthogonal to the longitudinal axis of the needle 10 and/or cannula 20. In one embodiment the biopsy robot is configured to control and adjust lateral directional positioning axis of the needle 10 and/or cannula 20 along a Y-axis orthogonal to the longitudinal axis of the needle 10 and/or cannula 20. In one embodiment the biopsy robot is configured to control and adjust lateral directional positioning axis of the needle 10 and/or cannula 20 in an X-Y plane orthogonal to the longitudinal axis of the needle 10 and/or cannula 20. In one embodiment the biopsy robot controls the positioning axis of the needle 10 and/or cannula 20 in a Z-axis along the longitudinal axis of the needle 10 and/or cannula 20. In various embodiments one or more motor units 500 can be mechanically and/or electronically connected to a cannula holding arm 510, an inner biopsy needle holding arm 520, a needle rotator 530, an inner needle grip 540, a cannula grip 550, and/or a needle pusher. In one embodiment the one or more motor units 500 are controlled through a wired system. In one embodiment the one or more motor units 500 are controlled through a wireless transmitter and remote control receiver 130.

In one embodiment a biopsy robot 570 has the case top 580 and case bottom 600. In one embodiment the case bottom 600 is wider than the case top 580. In one embodiment a biopsy robot 570 comprises a case top 580 and a case bottom 600 are connected by one or more legs 571. In one embodiment a biopsy robot case 570 the case top 580 and the case bottom 600 are connected by three legs, or tripod leg-shaped robot frames 571. In one embodiment a biopsy robot 570 comprises a housing made of any suitable material, such as plastic or metal. In one embodiment the housing is sealed. In one embodiment the housing is clear or transparent. In one embodiment the case bottom 600 is treated with one or more strong adhesives 610 that adhere the biopsy robot onto the patient skin for a good, tight fit to minimize movement between the patient and the biopsy robot. In one embodiment the adhesive treated extra cover adjacent to the case bottom 600 can be made of the flexible materials to tightly adhere to the skin without dead space.

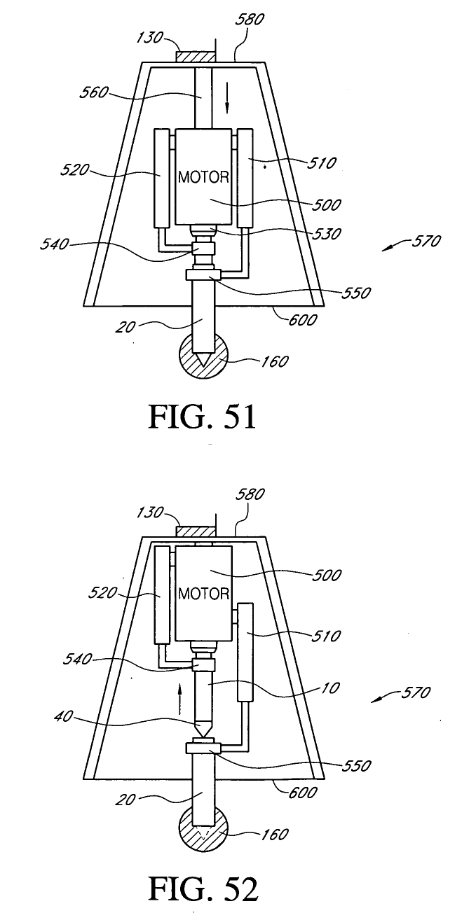

FIG. 49 illustrates an embodiment of the biopsy robot in a “pre-rotation” mode or configuration. In the pre-rotation mode the needle 10 and cannula 20 are in a proximal position. FIG. 50 is the perspective view of the biopsy robot in the “ready to rotate” mode. The needle pusher pushes the cannula 20 and the inner needle 10 distally into the target lesion 160. The needle 10 can be rotated to collect and hold a tissue sample. FIG. 51 is the perspective view of the biopsy robot when the inner biopsy needle 10 is pulled proximally backward after the cutting blades obtain the tissue sample 170 through a biopsy procedure, similar to the embodiment shown in FIGS. 21-25. After the confirmation of successful recovery of the biopsy tissue sample 170, the cannula holding arm 510 will pull back the cannula 20 from the patient body.

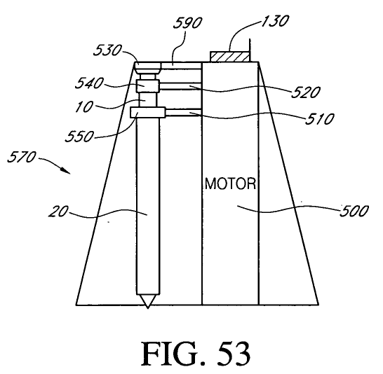

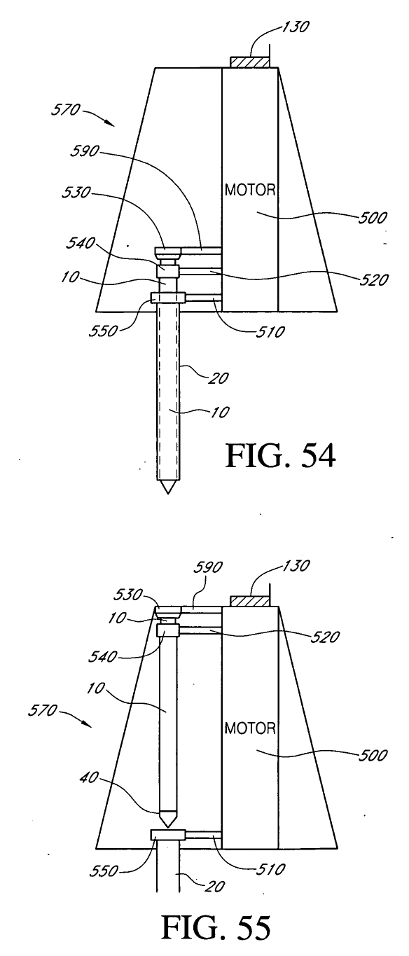

FIGS. 52-55 illustrate another embodiment of the biopsy robot in which the one or more motor units 500 is configured in a position roughly parallel to the cannula 20 and inner biopsy needle 10. The cannula holding arm 510, the inner needle holding arm 520 and the needle rotator holding arm 37 are controlled by the motor unit 500. This side-by side embodiment provides for a shorter overall robot height, or can allow for the use of a relatively longer biopsy needle 10. FIG. 53 shows the biopsy robot in the “ready to rotate” mode in which the cannula 20 and the inner biopsy needle 10 are inserted into the target lesion 160. FIG. 54 shows the inner biopsy needle 10 advancing into a tissue. FIG. 55 shows the inner biopsy needle 10 being pulled backward proximally by the inner needle holding arm 530 after the cutting blades 40 obtain the tissue sample 170 in a process similar to the embodiment shown in the FIGS. 21-25.

The above embodiments of this biopsy robot can be modified for biopsies of target lesions in various organs. For example, the biopsy needles are shorter and thicker for breast biopsy. Lung biopsy needles are much thinner and longer. More complicated biopsy robot can have the function to move more freely for needle localization in the x-y-z-direction by using more motor units. Therefore, the scope of this biopsy robot is not restricted to the above embodiments shown in the drawings. Using embodiments of the needle rotating mechanism and a wired or wireless remote control system, the whole biopsy procedure can allow a doctor perform the biopsy or surgical excision procedure completely hand free watching every moment of the movement of the needle and the patient in remote distance.

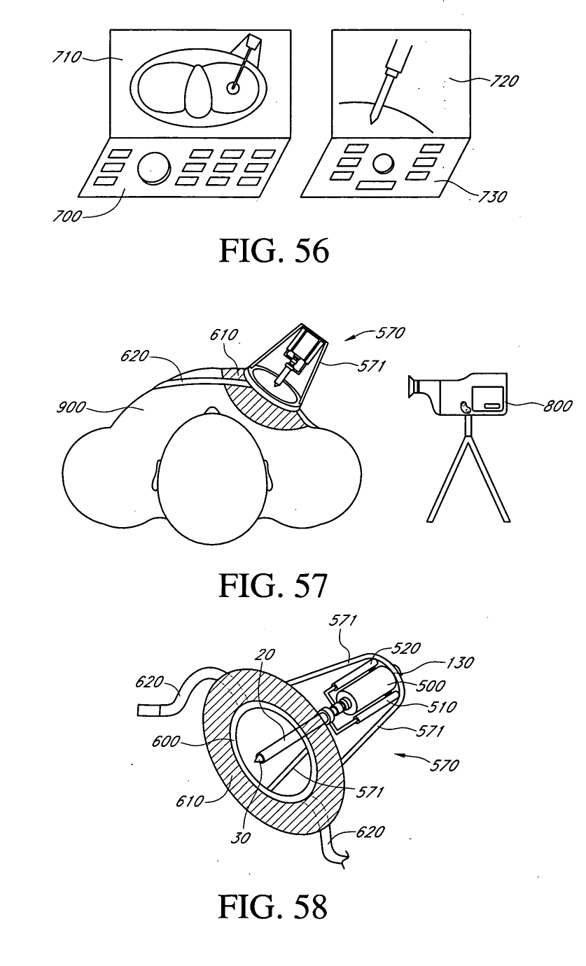

FIG. 56 illustrates one embodiment of an image display system comprising a CT image monitor 710, a video camera monitor 730, a CT image monitor control board 700 and a video camera monitory control board 730. In one embodiment a video camera 800 can show zoomed images of the biopsy needles and/or even the facial expression of the patient 900. In one embodiment a camera 800 is provided in the robot system. The imaging equipment can be of any type, such as ultrasound sonography, MRI, mammography, and others. Using the handheld controller (not shown in the drawing) the doctor can perform the biopsy procedure in a distance watching the images of the images, the biopsy needles and the patient at every moment of the procedure without needing to turn off the system to reduce radiation or other exposure when entering the operating room.

The complexity of embodiments of the biopsy robot use depending on the setting can be varied. For example, the relatively simple setting is that the doctor localizes the biopsy needles and pushes the needle rotator button 120 manually or by using the remote control system. The more complicated biopsy setting is that the doctor controls the biopsy robot in the control center from a remote distance. The doctor can communicate with the patient by watching the video camera images and perform the biopsy procedure using the biopsy robot.

FIG. 57 is a view of one embodiment of the uses of a portable biopsy robot of FIG. 58 attached to a patient 900. In one embodiment the bottom of the biopsy robot has the adhesive treated extended area 610 that adheres the robot to the patient's skin very tightly. In one embodiment the robot is securely attached to a patient's body by tying a strap 620 to help with the attachment of the robot to the patient 900. The patient 900 and the biopsy needle 10 can be monitored by the video camera 800. Several cameras can used so that the doctor can observe the patient and the needles in a close-up mode from different directions.

FIG. 58 illustrates an embodiment of the biopsy robot with a motor unit 500, the cannula 20, the conical sharp head of the inner biopsy needle 30, the cannula holding arm 510, the inner biopsy needle holding arm 520, the tripod leg shaped frames 571, the bottom of the biopsy robot 600, the adhesive treated extended area 610, and the strap 620 that wraps the robot to the patient's body 900.

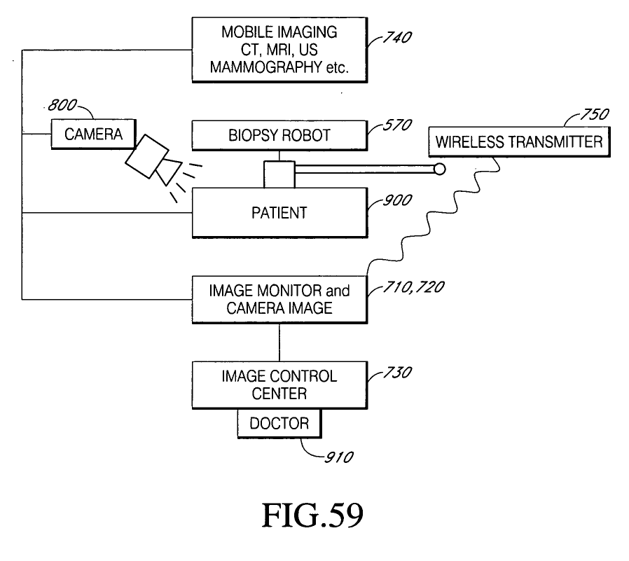

FIG. 59 is a block diagram of an embodiment of a wireless robotic biopsy system. The patient 900 has a biopsy robot 570 attached to the patient's body. In one embodiment the biopsy robot has a wireless transmitter 750 attached to communicate control and/or feedback signals between the robot 570 and a control center with one or more image monitors 710 and controls 700. In one embodiment a camera 800 is operatively linked to the control center with one or more image monitors 720 and controls 700. A doctor 910 can view and control the robot 570 through the monitors 710, 720 and control center 700, 730.

In one embodiment a distal portion of the blade 40 blade 221 can have a slightly sloped distal end blade 221, as is illustrated in FIG. 60. In one embodiment, the rotating biopsy device 1 can have a distal tip 231 at the distal end of blade 221. In one embodiment a distal portion of the blade 221 and/or the distal tip 231 can be slightly sloped in order to help move, displace, or penetrate through tissue more easily. In one embodiment a distal portion of the blade 221 is shaped like an arrowhead. In one embodiment the rotating blade 221 has a slightly sloped distal portion. In one embodiment the rotating blade 221 has a slightly concave front surface. In one embodiment the rotating blade 221 has a slightly convex front surface. In one embodiment the rotating blade 221 has a roughly straight front surface.

In some embodiments rotating blades 40, such as blade 221, are configured to function in various ways depending on direction of rotation and/or speed of rotation. The cutting angle, pitch, material, sharpness, and other features can be varied between first and second (or more) sides of a blade, thereby affecting the action of the blade depending on the direction and/or speed of rotation. In one embodiment the rotating blade 221 is configured to facilitate cutting of tissue at the distal end when the needle 241 is rotated in one of a first or second rotation direction. In one embodiment the rotating blade 221 is configured to facilitate cutting of hard tissue at the distal end when the needle 241 is rotated in one of a first or second rotation direction. In one embodiment the rotating blade 221 is configured to facilitate cutting of soft tissue at the distal end when the needle 241 is rotated in one of a first or second rotation direction. In one embodiment the rotating blade 221 is configured to facilitate displacing tissue without cutting or severing the tissue when the needle 241 is rotated in one of a first or second rotation direction. In one non-limiting example, a dull edge or surface can be presented on one side of the blade such that rotation in that direction moves tissues, while the other side of the blade can have a sharpened edge or surface to cut through tissue. For example, this non-cutting action may be similar in effect to using a stylet or guidewire by rotating the needle 10, 241 in the opposite of its cutting direction. In one embodiment, such a needle 241 can be used instead of using a separate stylet 180 and needle 10. The shape and configuration of the blades can be various depending on the type of biopsy or tissue excision.

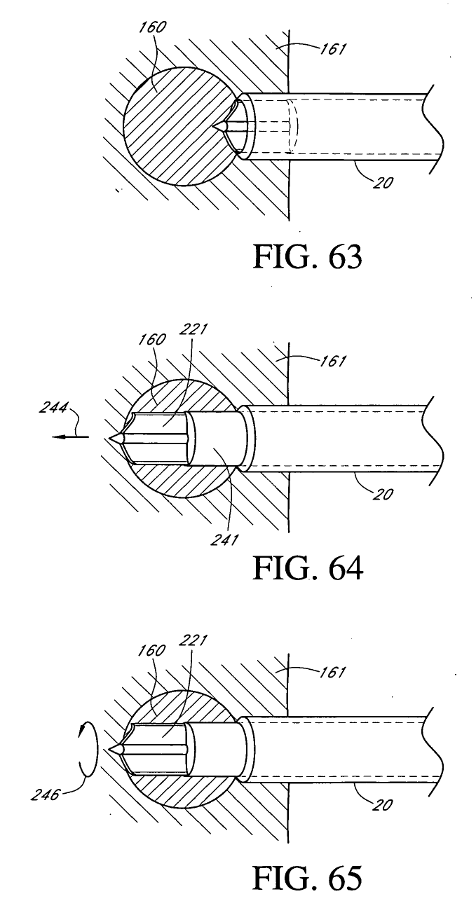

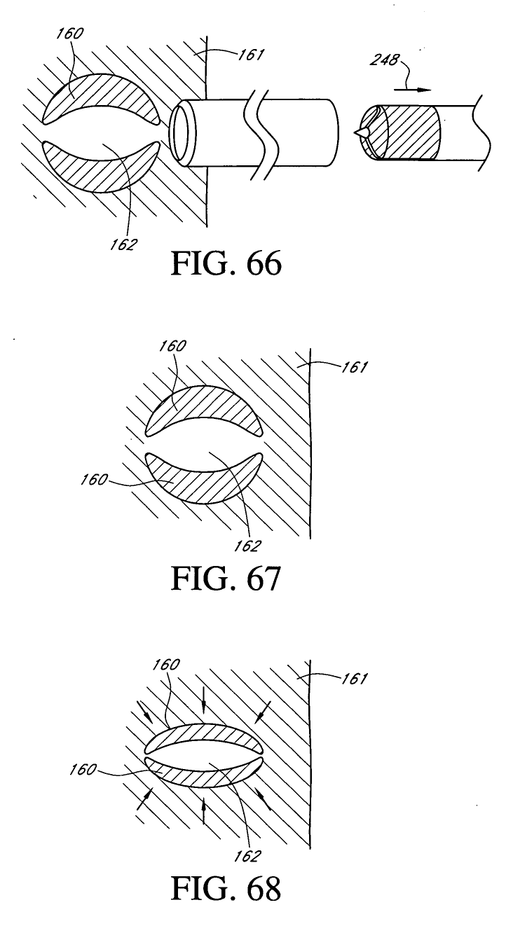

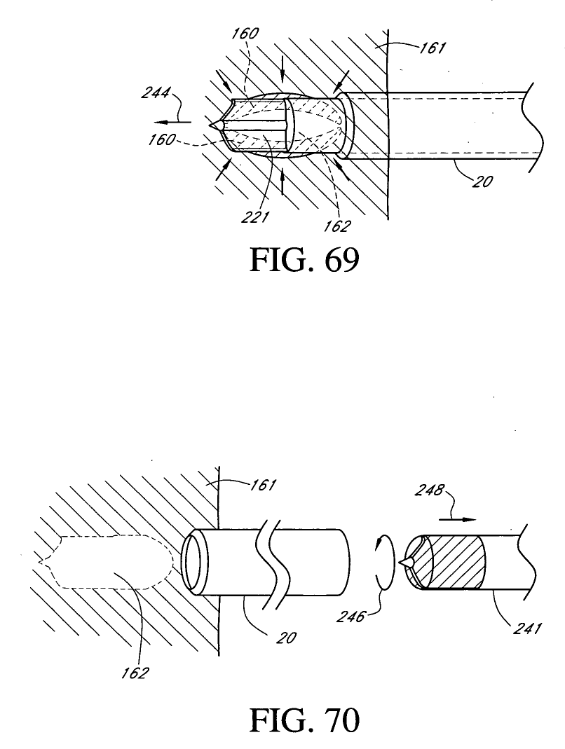

FIGS. 60-70 are perspective side views of an embodiment of a rotating biopsy device 1 with a rotating biopsy needle 241 and cannula 20. In the illustrated embodiment the rotating blade 221 is configured to facilitate cutting of tissue at the distal end when the needle 241 is rotated in a first direction 246. In the illustrated embodiment the rotating blade 221 is also configured to facilitate displacement of tissue when the needle 241 is rotated in a first or second direction 247. In one embodiment the rotation of the inner biopsy needle 241 and the blades 221 in a direction opposite to the cutting mode rotation direction, centrifugal force will be generated. This centrifugal force can be used to penetrate the tissue without cutting and injuring it. Thus, it can eliminate the need of a stylet 180 as described in the guide needle system above. In one embodiment rotation of the needle 241 in a first direction produces centrifugal force with a cutting action to hold a tissue sample in place on the blade. In one embodiment rotation in a second direction results in the surrounding tissues moving away from the rotating blades because of the configuration of the blade, such as in one non-limiting example, having a convex surface on the tissue moving side of the blade and a concave surface on the cutting side of the blade. In one embodiment the blade tip of the inner biopsy needle 241 in the cannula 20 can be advanced directly into the body by rotating in a direction opposite the cutting rotation direction until it reaches the target tissue 160, as illustrated in FIGS. 60-62. In one embodiment, once the rotating inner biopsy needle 241 reaches the target tissue 160, the rotation can be stopped as is illustrated in FIG. 63. In one embodiment illustrated in FIG. 64, the needle 241 is distally advanced in a direction 244. In one embodiment the needle 241 is advanced in distally axially in direction 244 to cut into or through the target tissue 160, as is illustrated in FIGS. 63 and 64. In one embodiment the needle 241 is advanced in distally in direction 244 while rotating in a cutting direction 246 into or through the target tissue 160. In one embodiment the needle 241 is rotated in a cutting direction 246 to cut the target tissue 160 to take a sample 170, leaving a cavity 162 in the tissue after the needle 241 is proximally withdrawn as illustrated in FIG. 66. In one embodiment the biopsy needle 241 continues to rotate while it is being withdrawn proximally, rotates centrifugally to keep the severed tissue 170 on at least a surface of at least one blade 221.

Depending on the size of the target tissue 160 to be removed in a sample 170, the needle 241 can be reinserted into the cannula 20 to access the target tissue 160 multiple times to get more samples. When sample 170 is removed from the target tissue 160 a cavity 162 remains, which can be at least partially collapsed by the surrounding tissue 161. Subsequent insertions in to the cannula 20 and sample removal by the rotating biopsy needle 241 can result in multiple samples from one or more target sites in the patient. This biopsy needle 1 can excise the target completely (complete excision) in addition to the biopsy function described above. In one embodiment, the biopsy needle 1 system can be used to completely excise target tissue through one or more repeated uses of the procedures described in the various embodiments of the methods described herein. In one embodiment a biopsy needle 1 can be used to excise the target completely when the target is too large to be excised at once. In one embodiment tissue can be excised by repeating the procedure as shown in FIGS. 66-70. Once the center of the target tissue 160 gets at least a portion sample 162 excised, it leaves a cavity 162. Due to a partial “vacuum effect” and the pressure by the surrounding tissue, the cavity 162 will collapse to certain extent, thus making the total dimension of the remaining target tissue 160 smaller. Then the repeated procedure(s) can remove the remaining target tissue 160 completely.

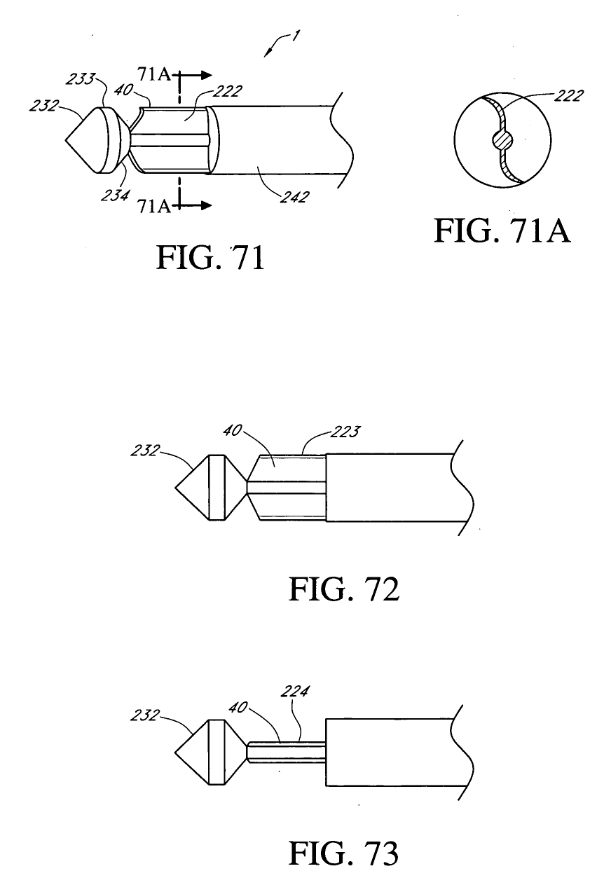

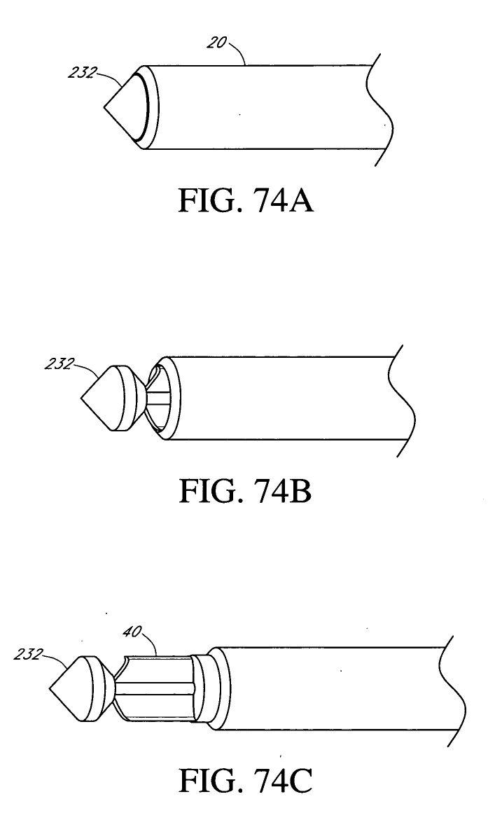

FIGS. 71-74C are perspective side and bottom views of an embodiment of a rotating biopsy device 1 with a cannula 20 and a rotating biopsy needle 242 with an extended distal tip 232. In one embodiment the rotating biopsy needle 242 has a blade 40 with an extended distal tip configuration blade 222. In one embodiment the rotating biopsy needle 242 with an extended distal tip 232 is configured to have a dual function, or multiple functions. One embodiment of the extended distal tip 232 can provide similar results as a stylet tip 180, such as pushing tissue out of the way of the advancing rotating biopsy device 1 to access a target tissue 160, without requiring a separate device that needs to be removed from the cannula 20 before a separate needle 10 is inserted. In one embodiment the extended distal tip 232 has a second surface 233 to help displace tissue 161 for the distal advancement of the rest of the needle 242. In one embodiment the second surface 233 is cylindrical to help form a tunnel-like shape in the surrounding tissue to create an access path for the rotating biopsy needle 242 and cannula 20. In one embodiment the extended distal tip 232 has a third surface 234 that tapers toward the central core of the needle 242 to provide room for tissue to be cut or displaced by the blades 40. In one embodiment the blade 40 is a tapered straight edged blade 223 with a diameter or width roughly equivalent to the diameter or width of the maximum diameter or width of the extended distal tip 232. FIG. 72 illustrates a side view of one embodiment of a rotating biopsy device 1 with a cannula 20 and a rotating biopsy needle 242 with an extended distal tip 232. In one embodiment the blade 40 with an extended distal tip configuration blade 222 has a rounded edge 223 for moving tissue without cutting tissue. FIG. 73 illustrates a bottom view of the embodiment of a rotating biopsy device 1 with a cannula 20 and a rotating biopsy needle 242 with an extended distal tip 232 as illustrated in FIG. 72. In one embodiment the blade 40 with an extended distal tip configuration blade 222 has a sharp edge 224 for cutting tissue. FIGS. 74A, 74B and 74C illustrate one embodiment of a rotating biopsy device 1 with a cannula 20 and a rotating biopsy needle 242 with an extended distal tip 232 as the extended distal tip 232 is moved distally from the cannula 20 to expose the blades 40.

In various embodiments of a rotating biopsy device 1, additional material, drugs or therapies can be delivered or administered through the cannula 20 before or after a sample 170 is taken with the needle. In one embodiment of a rotating biopsy device 1 chemotherapeutic drugs can be injected through the cannula lumen 21 before and/or after the excision to ensure complete elimination and eradication of cancer cells in the excision area. Anesthetic drugs such as lidocaine, or vaso-constricting agents such as epinephrine can be administered before or after the tissue sampling to numb the inner site or to control bleeding if necessary. In one embodiment, an infusion device 1000 delivers an infusion material to the target tissue 160 through the cannula 20. In one embodiment illustrated at FIG. 75, an infusion device 1000 delivers an infusion material to the cavity 162 through the cannula 20. In various embodiments, the infusion material can be a drug, such as chemotherapeutic drugs, or anesthetic drugs, or any material to be delivered to a target tissue site.

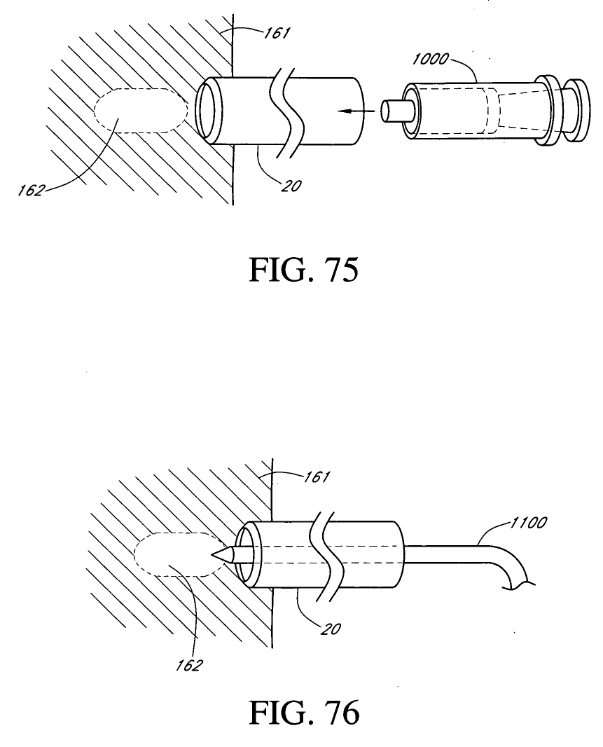

In one embodiment a target area treatment device 1100 delivers therapy to the target tissue 160 through the cannula 20. In one embodiment illustrated at FIG. 76, target area treatment device 1100 delivers a therapy to the cavity 162 through the cannula 20. In one embodiment the target area treatment device 1100 can include a radiofrequency ablation therapy device. In one embodiment the radiofrequency ablation therapy device burns the target tissue with microwaves delivered by a catheter or other wire system.

In one embodiment the target area treatment device 1100 can include a cryotherapy device that freezes target tissue. In various embodiments the target area treatment device 1100 can freeze the target tissue 160 to death or provide other chemical or physical methods to kill or weaken the target tissue 160.

It will be understood that the foregoing is only illustrative of the principles of the invention, and that various modifications, alterations, and combinations can be made by those skilled in the art without departing from the scope and spirit of the invention. Accordingly, it is not intended that the invention be limited, except as by the appended claims.

What is claimed is:

1. A rotating biopsy device for taking a tissue sample from a target tissue site in a patient body, comprising:
a cannula with a lumen, the cannula configured to define an access path to the target tissue site; and
a rotational biopsy needle with at least one blade, the rotational biopsy needle axially and rotatably moveable within the cannula lumen, the at least one blade configured to remove a tissue sample from the target tissue site through rotation of the at least one blade, the at least one blade configured to hold the tissue sample in the rotational biopsy needle during proximal retraction from the patient.

2. The rotating biopsy device of claim 1, the rotational biopsy needle further comprising a sharp distal head.

3. The rotating biopsy device of claim 1, the at least one blade further comprising a first surface and a second surface, at least one of the first surface and second surface configured to retain a tissue sample.

4. The rotating biopsy device of claim 3, the at least one blade further comprising a first edge.

5. The rotating biopsy device of claim 1, wherein the rotational biopsy needle further comprises a locking mechanism to releasably lock the rotational biopsy needle position with respect to the cannula.

6. The rotating biopsy device of claim 1, further comprising a guide needle.

7. The rotating biopsy device of claim 1, further comprising a needle rotator.

8. The rotating biopsy device of claim 7, wherein the needle rotator comprises a motor.

9. The rotating biopsy device of claim 7, wherein the needle rotator comprises a remote control.

10. The rotating biopsy device of claim 1, further comprising a biopsy robot with an adhesive configured to adhere the biopsy robot to the patient's body.

11. The rotating biopsy device of claim 1, further comprising a biopsy robot with a strap configured to attach the biopsy robot to the patient's body.

12. A method of collecting a tissue sample from a target tissue site in a body of a patient, comprising:
inserting a rotational biopsy needle with at least one blade in a patient, the rotational biopsy needle axially and rotatably moveable within a lumen of a cannula, the at least one blade configured to cut a tissue sample from the target tissue site through rotation of the at least one blade, the at least one blade configured to hold the tissue sample in the rotational biopsy needle during proximal retraction from the patient;
distally advancing the rotational biopsy needle to a target tissue site in the patient;
rotating the rotational biopsy needle in a first direction to remove a tissue sample from the target tissue site;
holding a removed tissue sample from the target tissue site on the at least one blade; and
proximally retracting the rotational biopsy needle out of the body of the patient.

13. The method of collecting a tissue sample of claim 12, further comprising inserting the cannula in a patient's body to provide an access path for the rotational biopsy needle.

14. The method of collecting a tissue sample of claim 13, further comprising locking the cannula to the rotational biopsy needle prior to insertion into the patient's body.

15. The method of collecting a tissue sample of claim 12, further comprising adjusting the lateral direction of the rotational biopsy needle in a direction orthogonal to the longitudinal axis of the rotational biopsy needle.

16. The method of collecting a tissue sample of claim 12, further comprising attaching a biopsy robot to the patient's body.

17. The method of collecting a tissue sample of claim 12, further comprising reinserting the rotational biopsy needle to remove an additional tissue sample from the target tissue site.

18. The method of collecting a tissue sample of claim 12, further comprising completely excising the target tissue site.

19. The method of collecting a tissue sample of claim 12, further comprising rotating the rotational biopsy needle in a second direction opposite the first direction to facilitate the distal advancement of the rotational biopsy needle to the target tissue site.

20. The method of collecting a tissue sample of claim 12, further comprising rotating the rotational biopsy needle in a second direction opposite the first direction to remove the tissue sample from the rotational biopsy needle.

21. The method of collecting a tissue sample of claim 12, further comprising infusing a material to the target tissue site with an infusion device.

22. The method of collecting a tissue sample of claim 12, further comprising treating the target tissue site with a target area treatment device.

23. A biopsy robot, comprising:
a cannula with a lumen, the cannula configured to access the target tissue site;
a rotational biopsy needle with at least one blade, the rotational biopsy needle axially and rotatably moveable within the cannula lumen, the at least one blade configured to separate a tissue sample from the target tissue site through rotation of the at least one blade, the at least one blade configured to hold the tissue sample in the rotational biopsy needle during proximal retraction from the patient;
a motor; and
a controller.

24. The biopsy robot of claim 23, further comprising a case bottom with an adhesive configured to adhere the biopsy robot to the patient's body.

25. The biopsy robot of claim 23, wherein the controller is controlled from a remote location.
